(19) United States
(12) Patent Application Publication
(10) Pub. No.: US 2020/0237852 A1
(43) Pub. Date: Jul. 30, 2020

(54) Small Molecule Enhancers of Paneth Cell Function and Differentiation

(71) Applicant: Massachusetts Institute of Technology, Cambridge, MA (US)

(72) Inventors: Benjamin Elliott Mead, Cambridge, MA (US); Lauren Levy, Cambridge, MA (US); Daphne Sze, Morris Plains, NJ (US); Robert Samuel Langer, Jr., Newton, MA (US); Jeffrey Michael Karp, Brookline, MA (US); Kazuki Hattori, Cambridge, MA (US); Alexander Kann Shalek, Cambridge, MA (US)

(21) Appl. No.: 16/698,513

(22) Filed: Nov. 27, 2019

Related U.S. Application Data
(60) Provisional application No. 62/799,025, filed on Jan. 30, 2019.

(51) Int. Cl. A61K 38/06; A61K 31/497; A61K 31/506; A61K 31/366

(52) U.S. Cl. CPC A61K 38/06; A61K 31/366; A61K 31/506; A61K 31/497

(57) ABSTRACT

Leucine-rich repeat-containing G-protein coupled receptor 5-positive (LGR5⁺) intestinal cells are contacted with an inhibitor of exportin 1 (XPO1), thereby producing functionally differentiated intestinal cells. The LGR5⁺ cells can also be contacted with a Wnt agonist. The LGR5⁺ cells can also be contacted with a Notch inhibitor.

### RELATED APPLICATION

This application claims the benefit of U.S. Provisional Application No. 62/799,025 filed on Jan. 30, 2019. The entire teachings of the above application are incorporated herein by reference.

### GOVERNMENT SUPPORT

This invention was made with Government support under Grant Nos. R01 DE013023 and R01 HL094722 awarded by the National Institutes of Health (NIH). The Government has certain rights in the invention.

### BACKGROUND

The intestinal epithelium is a complex tissue that plays a key role in digestion and mediates innate and adaptive immune functions. The small intestinal epithelium is formed by a single layer of cells arranged into villi—primarily composed of enterocytes, absorptive cells, and secretory Goblet cells—and crypts, which contain intestinal stem cells (ISCs) and secretory Paneth cells (PCs). Intestinal stem cells differentiate into mature intestinal cells, but signaling pathways and factors that modulate differentiation to Paneth cells are insufficiently understood. Several inflammatory and disease states are associated with intestinal irregularities, including inflammatory bowel disease, Crohn's disease, necrotizing enterocolitis, and intestinal inflammation.

### SUMMARY

Described herein is a method of differentiating leucine-rich repeat-containing G-protein coupled receptor 5-positive (LGR5⁺) intestinal cells. The method can include contacting LGR5⁺ cells with an inhibitor of exportin 1 (XPO1), thereby producing functionally differentiated intestinal cells. Inhibitors of XPO1 include KPT-330, KPT-8602, and Leptomycin B.

In some embodiments, the method further includes contacting the LGR5⁺ cells with a Wnt agonist, such as CHIR99021. In some embodiments, the method further includes contacting the LGR5⁺ cells with a Notch inhibitor, such as DAPT.

The functionally differentiated intestinal cells can be Paneth cells. The functionally differentiated intestinal cells can be CD24-mid/LYZ⁺ cells. The LGR5⁺ intestinal cells can be intestinal stem cells.

In some embodiments, the functionally differentiated intestinal cells can secrete greater quantities of lysozyme compared to the LGR5⁺ intestinal cells. In some embodiments, the functionally differentiated intestinal cells express any combination of one or more of human lysozyme (LYZ), a human alpha defensin (DEFA), human matrix metalloproteinase-7 (MMP-7), and cluster of differentiation 24 (CD24). In some embodiments, the functionally differentiated intestinal cells express human lysozyme (LYZ). In some embodiments, the functionally differentiated intestinal cells can express a human alpha defensin (DEFA), such as human alpha defensin 5 (DEFA5) or human alpha defensin 6 (DEFA6).

In some embodiments, the functionally differentiated intestinal cells express mRNA for any combination of one or more of human lysozyme (LYZ), a human alpha defensin (DEFA), human matrix metalloproteinase-7 (MMP-7), and cluster of differentiation 24 (CD24). In some embodiments, the functionally differentiated intestinal cells express mRNA for human lysozyme (LYZ). In some embodiments, the functionally differentiated intestinal cells express mRNA for a human alpha defensin (DEFA), such as mRNA for human alpha defensin 5 (DEFA5) or mRNA for human alpha defensin 6 (DEFA6).

Described herein is a method of producing functionally differentiated intestinal cells. The method can include obtaining intestinal cells from an individual; contacting the intestinal cells with a Wnt agonist and a histone deacetylase (HDAC) inhibitor to produce LGR5⁺ intestinal cells; and contacting the LGR5⁺ intestinal cells with a Wnt agonist, a Notch inhibitor, and an inhibitor of exportin 1 (XPO1), thereby producing functionally differentiated intestinal cells. The HDAC inhibitor can be valproic acid. The method can further include contacting the LGR5⁺ cells with a Wnt agonist. The method can further include contacting the LGR5+ cells with a Notch inhibitor.

Described herein is a cell culture solution that includes a Wnt agonist, a Notch inhibitor, and an inhibitor of exportin 1 (XPO1).

Described herein are methods of treating diseases in an individual in need thereof. Examples of diseases include graft-versus-host disease, inflammatory bowel disease, Crohn's disease, and necrotizing enterocolitis. The methods can also reduce intestinal inflammation (e.g., epithelial inflammation; intestinal inflammation hat occurs within epithelial cells) in an individual in need thereof. The methods can include administering an effective amount of an inhibitor of exportin 1 (XPO1) to intestinal cells of the individual. In some embodiments, the methods further include contacting the intestinal cells with a Wnt agonist. In some embodiments, the methods further include contacting the intestinal cells with a Notch inhibitor.

Described herein is a method of increasing secretion of an antimicrobial protein in intestinal cells. The method can include contacting LGR5⁺ intestinal cells with an inhibitor of exportin 1 (XPO1). The antimicrobial protein can be one or more of human lysozyme (LYZ), human regenerating islet-derived 3 gamma (REG3G), and a human alpha-defensin (DEFA).

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A: Schematic outlining the alterations in culture format between traditional “3-D” organoid culture and “2.5-D” culture, which enables the enhanced multiplexed measurement of secreted supernatant lysozyme and cell pellet adenosine adenosine-5′-triphosphate (ATP).

FIG. 1B: Supernatant Lyz measurements of 6-day ENR+CD cell cultures plated in 96-well plates in either 3-D or 2.5-D format following basal or 10 μM CCh stimulation from 12-well replicates (2 bio. donors), error bars S.E.M., *adj. p<0.05, ****adj. p<0.0001. FIG. 1C: Supernatant Lyz and cell pellet ATP measurements from 6-day ENR+CD cell cultures in 2.5-D 96-w plates from 12-well replicates (3 bio. donors), error bars S.E.M. FIG. 1D: Supernatant Lyz normalized to matched-well ATP from 6-day ENR+CD cell cultures in 2.5-D 96-w plates from 12-well replicates (3 bio. donors), error bars S.E.M., with matched coefficient or variation over varying cell-cluster density per well. FIG. 1E: Supernatant Lyz normalized to matched-well ATP from 6-day ENR, ENR+CV, ENR+CD cell cultures in 2.5-D 96-w plates from 8-well replicates (2 bio. donors), error bars S.E.M., **adj. p<0.01, ****adj. p<0.0001.

FIG. 2A: Schematic of scalable 2.5-D platform for assessing short-term (3-hour) modulators of 6-day ENR+CD LYZ secretion. FIG. 2B: UMVUE strictly standardized mean difference (SSMD) following 3-hour stimulation of 6-day ENR+CD for LYZ secretion, 9-well replicates from 3 bio. donors. FIG. 2C: Supernatant LYZ least-squares-fit dose-response curve for CCh stimulation of 6-day ENR+CD cells, 9-well replicates from 3 bio. donors, error bars S.E.M. FIG. 2D: Cell pellet ATP dose-response curve for matched points in (C), **adj. p<0.01. FIG. 2E: Supernatant LYZ least-squares-fit dose-response curve for LPS stimulation of 6-day ENR+CD cells, 9-well replicates from 3 bio. donors, error bars S.E.M. FIG. 2F: Cell pellet ATP dose-response curve for matched points in (E), ns: adj. p>0.05. FIG. 2G: Supernatant LYZ least-squares-fit dose-response curve for IFN-γ stimulation of 6-day ENR+CD cells, 9-well replicates from 3 bio. donors, error bars S.E.M. FIG. 2H: Cell pellet ATP dose-response curve for matched points in (G), ns: adj. p>0.05.

FIG. 3A: Schematic for assessing long-term (48-hour) modulators of 6-day ENR+CD LYZ secretion. FIG. 3B: Log-scale fold change in ATP (rel. to ENR+CD) for long-term (48-hour) treatments with corresponding SSMD below from 32 well replicates from 4 bio. donors, error bars S.E.M. FIG. 3C: Log-scale fold change in ATP (rel. to ENR+CD) for long-term (48-hour) treatments with corresponding SSMD below from 16 well replicates from 4 bio. donors, error bars S.E.M. FIG. 3D: Log-scale fold change in ATP (rel. to ENR+CD) for long-term (48-hour) treatments with corresponding SSMD below from 16 well replicates from 4 bio. donors, error bars S.E.M.

FIG. 4A: Schematic for in vitro 6-day PC differentiation screen and multiplexed single-well assays. FIG. 4B: Replicate UMVUE SSMD for each well and assay in screen, colored points are deemed hits above FPL and FNL-determined cutoff, circled points are hits in both LYZ.NS and LYZ.S assays, each point represents the SSMD from 3 replicates of 3 bio. donors relative to whole-plate control. FIG. 4C: Venn diagram of treatment hits based on replicate SSMD across the 3 assays. FIG. 4D: Mean fold change of assay effect for hits in LYZ.S and LYZ.NS, only points above 1.28 standard deviations of all treatment mean fold changes (corresponding to the top 10% of a normal distribution) are deemed potent hits. FIG. 4E: Table of all potent LYZ.NS and LYZ.S assay hits from screen, separated by characterized pathways of compound activity.

FIG. 5A: Distribution of all sample data (n=5676 wells) for each assay following data transformation and normalization, dotted line indicates median of distribution for which all fold change calculations are determined. FIG. 5B: ATP assay controls across all plates and replicates, **** p<0.0001. FIG. 5C: LYZ.NS assay controls across all plates and replicates, **** adj. p<0.0001. FIG. 5D: ATP assay controls across all plates and replicates, * adj. p<0.05, **** p<0.0001. FIG. 5E: Spearman correlation (r) between all sample wells by screen plate and biological replicate.

FIG. 6A: Fold-change (log 10) response relative to whole plate for TGF-beta inhibitor hits for ATP, and secreted LYZ assays (CCh stimulated—LYZ.S and basal—LYZ.NS), error bars S.E.M., n=3. FIG. 6B: Fold-change (log 10) response relative to whole plate for PI3K/Akt/mTOR inhibitor hits for ATP, and secreted LYZ assays (CCh stimulated—LYZ.S and basal—LYZ.NS), error bars S.E.M., n=3. FIG. 6C: Fold-change (log 10) response relative to whole plate for Tyr kinase inhibitor hits for ATP, and secreted LYZ assays (CCh stimulated—LYZ.S and basal—LYZ.NS), error bars S.E.M., n=3. FIG. 6D: Fold-change (log 10) response relative to whole plate for other inhibitor hits for ATP, and secreted LYZ assays (CCh stimulated—LYZ.S and basal—LYZ.NS), error bars S.E.M., n=3.

FIG. 7A: SSMD for LYZ.NS, LYZ.S, and ATP assays for 6-day ENR+CD+treatment versus ENR+CD+DMSO (vehicle) control, with an FPL=0.05 determined cutoff (0.89), colored dots signify treatment-doses passing cutoffs for both LYZ assays, n=8 well replicates. FIG. 7B: Biological potency for LYZ.NS versus LYZ.S assays based on mean fold change (based on n=8 well replicates) of treatment relative to control, red signifies treatments advanced for profiling. FIG. 7C: Table of validated small molecules at their maximal doses, color-coded per FIG. 4E. FIG. 7D: Schematic and fold-change (FC) results for early (day 0-3) vs. full (day 0-6) treatment of validated compounds relative to control in LYZ.NS, LYZ.S, and ATP assays, error bars S.E.M., n=4 early and n=8 full, * adj. p<0.05. FIG. 7E: Flow cytometry profiling of mature PC (CD24-mid & LYZ+) and secretory precursor (CD24-hi & LYZ−) populations as a fraction of all viable cells after 6-day ENR+CD+treatment culture, n=3 bio. donor replicates, error bars S.E.M., ** adj. p<0.01.

FIG. 8: Fold change (FC) for early (day 0-3) vs. full (day 0-6) treatment of ENR+CD cells at all doses for each validated compound relative to ENR+CD control in LYZ.NS normalized to ATP, LYZ.S normalized to ATP, and ATP assays, error bars S.E.M., n=4 early and n=8 full, * adj. p<0.05.

FIG. 9A: SSMD for ATP, LYZ.NS, and LYZ.S assays for 6-day ENR+treatment vs. ENR+DMSO (vehicle) control, with an FPL=0.05 determined cutoff (−0.89), colored dots signify treatment-doses passing cutoffs for both LYZ assays in ENR+CD (FIG. 7A), circle dots pass negative cutoffs in ENR, n=8 well replicates. FIG. 9B: Mean fold change (m.f.c. for n=8 well replicates) for ENR vs. ENR+CD relative to respective controls at each tested dose for select validated compounds in LYZ.NS normalized to ATP, LYZ.S normalized to ATP assays. FIG. 9C: Mean fold change (m.f.c. for n=8 well replicates) for ENR vs. ENR+CD relative to respective controls at each tested dose for select validated compounds in ATP assay.

FIG. 10A: Mean fold change (m.f.c. for n=8 well replicates) for ENR vs. ENR+CD relative to respective controls at each tested dose for remaining (not in FIG. 9B) validated compounds in LYZ.NS normalized to ATP, LYZ.S normalized to ATP assays. FIG. 10B: Mean fold change (m.f.c. for n=8 well replicates) for ENR vs. ENR+CD relative to respective controls at each tested dose for remaining (not in FIG. 9C) validated compounds in ATP assay.

FIGS. 11A-D pertain to western blotting and LYZ secretion assay following XPO1 inhibition in ISC differentiation. FIG. 11A: Western blotting for LYZ in organoids cultured in ENR+CV or ENR with or without XPO1 inhibitors for six days. FIG. 11B: Western blotting for LYZ in organoids cultured in ENR+CD with or without XPO1 inhibitors for six days. FIG. 11C: Lysozyme secretion assay normalized by whole-well ATP, conducted on organoids differentiated in ENR+CD for 6 days with multiple XPO1 inhibitors, with both induced (CCh—carbamyl choline) and non-induced secretion. Dunnett's multiple comparison test: ** adj. p<0.01, *** adj. p<0.005, n=5 well replicates. FIG. 11D: Lysozyme secretion assay normalized by whole-well ATP, conducted on organoids differentiated in ENR for 6 days with multiple XPO1 inhibitors, with both induced (CCh—carbamyl choline) and non-induced secretion. Dunnett's multiple comparison test: ** adj. p<0.01, **** adj. p<0.0001, n=8 well replicates.

FIGS. 12A-E pertain to Population RNA-seq following XPO1 and mixed Wnt/Notch signaling changes during ISC differentiation. FIG. 12A: Outline of conditions sampled in population RNA-seq experiment, n=4 well replicates for all conditions. FIG. 12B: Module scoring using in vivo Paneth cell-defining genes for ENR+CD+/−XPO1 inhibitor-treated organoids (ENR as reference), * adj. p<0.05. FIG. 12C: Module scoring using in vivo Paneth cell-defining genes for ENR+/−XPO1 inhibitor-treated organoids (ENR+CD as reference), * adj. p<0.05, *** adj. p<0.001. FIG. 12D: Module scoring using in vivo enteroendocrine cell-defining genes for ENR+CD+/−XPO1 inhibitor-treated organoids, * adj. p<0.05, *** adj. p<0.001. FIG. 12E: Effect size model over all media conditions at day 3 of treatment for KPT-330 and KPT-8602 versus no treatment, with differentially regulated genes (FDR<0.01) shown, colored by defining Paneth (blue) and enteroendocrine (purple) genes.

FIG. 13 is a graph and flow diagram pertaining to the flow cytometry gating strategy used to identify mature Paneth cells, namely as a population with high FITC-LYZ expression and intermediate APC-CD24 expression in a system where secretory precursors may be present (APC-CD24 high expression). Additionally, side scatter as a measure of intercellular granularity (SSC) can meaningfully discriminate granule-dense Paneth cells.

FIG. 14 is charts pertaining to measurements of the Paneth cell composition via flow cytometry of ENR+CD organoids treated with the known small molecule XPO1 inhibitors KPT-330, KPT-8602, and Leptomycin B.

### DEFINITIONS

As used herein, “or” means “and/or” unless stated otherwise.

As used herein, the term “comprise” and variations of the term, such as “comprising” and “comprises,” are not intended to exclude other additives, components, integers or steps.

As used herein, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Administration” refers to introducing a substance into a subject. In some embodiments, administration is oral, or by injection. In certain embodiments “causing to be administered” refers to administration of a second component after a first component has already been administered (e.g., at a different time and/or by a different actor).

An “antibody” refers to an immunoglobulin polypeptide, or fragment thereof, having immunogen binding ability.

As used herein, an “agonist” is an agent that causes an increase in the expression or activity of a target gene, protein, or a pathway, respectively. An agonist can bind to and activate its cognate receptor in some fashion, which directly or indirectly brings about this physiological effect on the target gene or protein. An agonist can also increase the activity of a pathway through modulating the activity of pathway components, for example, through inhibiting the activity of negative regulators of a pathway. Therefore, a “Wnt agonist” can be defined as an agent that increases the activity of Wnt pathway, which can be measured by increased TCF/LEF-mediated transcription in a cell. Therefore, a “Wnt agonist” can be a true Wnt agonist that bind and activate a Frizzled receptor family member, including any and all of the Wnt family proteins, an inhibitor of intracellular beta-catenin degradation, and activators of TCF/LEF. A “Notch agonist” can be defined as an agent that increase the activity of Notch pathway, which can be determined by measuring the transcriptional activity of Notch.

An “antagonist” refers to an agent that binds to a receptor, and which in turn decreases or eliminates binding by other molecules.

“Cell differentiation” refers to the process by which a cell becomes specialized to perform a specific function, such as in the conversion of post-natal stem cells into cells having a more specialized function. In embodiments, LGR5⁺ intestinal stem cells are differentiated into Paneth cells or cells that express characteristics of Paneth cells.

“CD” refers to CHIR99021 (C) and DAPT (D).

“Decreasing” and “decreases” refer to decreasing by at least 5%, for example, 5, 6, 7, 8, 9, 10, 15, 20, 25, 30, 35, 40, 45, 50, 55, 60, 65, 70, 75, 80, 85, 90, 95, 99 or 100%, for example, as compared to the level of reference, and includes decreases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a reference.

“Eliminate” means to decrease to a level that is undetectable.

“Enteroendocrine cells” refers to cells that are specialized endocrine cells of the gastrointestinal tract and pancreas, and can be found in the intestinal tract, stomach and pancreas. The enteroendocrine cells form the largest endocrine system in the body, and can sense luminal contents, particularly nutrients, and respond by the secretion of a diversity of hormones (e.g. GLP-1) which modulate food intake, energy homeostasis and glucose tolerance. Specific types of enteroendocrine cell are often classified according to the expression of hormones within the specific enteroendocrine cell subset, such as cells that express GLP-1, SHT, SST, gastrin, CCK, SCT, NTS, PYY, Gastrin and Ghrelin, among others. The different subsets of enteroendocrine have also been sometimes referred to as K cells, I cells, L cells, G cells, Enterochromaffin cells, N cells and S cells, but increasingly the hormone expression of the cells is used to identify the cell subtypes, as set forth above. Enteroendocrine cells can be identified by expression of ChgA marker.

“ENR” refers to epidermal growth factor (EGF) (E), noggin (N) and R-spondin 1 (R).

“Growth factor” refers to a substance capable of stimulating cellular growth, proliferation or differentiation.

“GSK3beta,” “GSK3β,” and “GSK3B” as used interchangeably herein are acronyms for glycogen synthase kinase 3 beta.

“GSK3beta inhibitor” is a substance that inhibits the activity of GSK3beta.

“HDAC” is an acronym for histone deacetylase, which is an enzyme that deacetylases histone proteins.

“HDAC inhibitor” is a substance that inhibits the activity of an HDAC.

An “inhibitor” refers to an agent that causes a decrease in expression or activity, e.g., of a target gene, a protein, or a pathway. For example, an “Wnt inhibitor” refers an agent that causes a decrease in the activity of Wnt signaling pathway, which can be for example a Wnt receptor inhibitor, a Wnt receptor antagonist, a Porcupine inhibitor which inhibits Wnt secretion, or a Tankyrase inhibitor, or a drug that interferes with β-catenin interactions. An “antagonist” can be an inhibitor, but is more specifically an agent that binds to a receptor, and which in turn decreases or eliminates binding by other molecules.

“Increasing” and “increases” refer to increasing by at least 5%, for example, 5, 6, 7, 8, 9, 10, 15, 20, 25, 30, 35, 40, 45, 50, 55, 60, 65, 70, 75, 80, 85, 90, 95, 99, 100% or more, for example, as compared to the level of a reference, and includes increases by at least 1-fold, for example, 1, 2, 3, 4, 5, 6, 7, 8, 9, 10, 15, 20, 30, 40, 50, 60, 70, 80, 90, 100, 200, 500, 1000-fold or more, for example, as compared to the level of a reference standard.

“Intestinal stem cell” refers to a multipotent cell of intestinal lineage which has the potential to become committed to multiple cell lineages, including cell lineages resulting in intestinal cells such as enteroendocrine cells, enterocyte cells, goblet cells and Paneth cells.

“LGR5” is an acronym for the leucine-rich repeat-containing G-protein coupled receptor 5, also known as G-protein coupled receptor 49 (GPR49) or G-protein coupled receptor 67 (GPR67). It is a protein that in humans is encoded by the Lgr5 gene.

“LGR5+ cell” or “LGR5-positive cell” is a cell that expresses Lgr5.

“Mammal” refers to any mammal including but not limited to human, mouse, rat, sheep, monkey, goat, rabbit, hamster, horse, cow or pig.

A “multipotent cell” refers to refers to a cell that is capable of differentiating into multiple different, but limited cell types.

“Non-human mammal,” as used herein, refers to any mammal that is not a human.

“Notch inhibitor” refers to an inhibitor of the Notch signaling pathway.

As used in relevant context herein, the term “number” of cells can be 0, 1, or more cells.

“Organoid” refers to a cell cluster or aggregate that resembles an organ, or part of an organ, and possesses cell types relevant to that particular organ.

“PC” refers to a Paneth cell or Paneth cells, as appropriate under the circumstances.

“Pharmaceutically acceptable” refers to those compounds, materials, compositions, and/or dosage forms which are, within the scope of sound medical judgment, suitable for use in contact with the tissues of human beings and non-human animals without excessive toxicity, irritation, allergic response, or other problem or complication, commensurate with a reasonable benefit/risk ratio.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. For example, inorganic salts include, but are not limited to, ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Example organic bases used in certain embodiments include isopropylamine, diethylamine, ethanolamine, trimethylamine, dicyclohexylamine, choline and caffeine.

“Population” of cells refers to any number of cells greater than 1, and even at least 1×10³ cells, at least 1×10⁴ cells, at least at least 1×10⁵ cells, at least 1×10⁶ cells, at least 1×10⁷ cells, at least 1×10⁸ cells, at least 1×10⁹ cells, or at least 1×10¹⁰ cells.

“Post-natal cell” refers to a non-embryonic cell.

“Post-natal stem cell” refers to non-embryonic stem cells that have the capacity to self-renew and to differentiate into multiple cell lineages. Post-natal stem cells may also be referred to as adult stem cells or somatic stem cells. Post-natal stem cells can include intestinal stem cells, epithelial stem cells, hematopoietic stem cells, mammary stem cells, mesenchymal stem cells, endothelial stem cells and neural stem cells.

“Progenitor,” “progenitor cell,” and “progenitor population,” as used herein refers to a cell (or cell population) that, like a stem cell, has the tendency to differentiate into a specific type of cell, but is already more specific than a stem cell and is pushed to differentiate into its “target” cell.

“Reference” means a standard or control condition (e.g., untreated with a test agent or combination of test agents).

“Sample” refers to a volume or mass obtained, provided, and/or subjected to analysis. In some embodiments, a sample is or comprises a tissue sample, cell sample, a fluid sample, and the like. In some embodiments, a sample is taken from (or is) a subject (e.g., a human or non-human animal subject). In some embodiments, a tissue sample is or comprises brain, hair (including roots), buccal swabs, blood, saliva, semen, muscle, or from any internal organs, or cancer, precancerous, or tumor cells associated with any one of these. A fluid may be, but is not limited to, urine, blood, ascites, pleural fluid, spinal fluid, and the like. A body tissue can include, but is not limited to, brain, skin, muscle, endometrial, uterine, and cervical tissue or cancer, precancerous, or tumor cells associated with any one of these.

“Self-renewal” refers to the process by which a stem cell divides to generate one (asymmetric division) or two (symmetric division) daughter cells with development potentials that are indistinguishable from those of the mother cell. Self-renewal involves both proliferation and maintenance of an undifferentiated state.

“Small molecule” as referred to herein refers to an organic compound that can participate in regulating biological pathways, and is a non-nucleic acid, is typically non-peptidic and non-oligomeric, and may have a molecular weight of less than 1500 daltons.

“Stem cell” refers to a multipotent cell having the capacity to self-renew and to differentiate into multiple cell lineages.

“Subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals, particularly pets such as dogs and cats. In some embodiments (e.g., particularly in research contexts) subject mammals will be, for example, rodents (e.g., mice, rats, hamsters), rabbits, primates, or swine such as inbred pigs and the like.

“Synergy” or “synergistic effect” is an effect which is greater than the sum of each of the effects taken separately; a greater than additive effect.

“TgfBeta inhibitor” and “Tgf-β inhibitor” refer to a substance that reduces activity of the TgfBeta pathway. An example of a TgfBeta inhibitor can be a TgfBeta receptor inhibitor, which may include but is not limited to Alk4, Alk7 and Alk5/TgfBeta-RI.

“Tissue” is an ensemble of similar cells from the same origin that together carry out a specific function.

“Treating” in connection with a cell population means delivering a substance to the population to effect an outcome. In the case of in vitro populations, the substance may be directly (or even indirectly) delivered to the population. In the case of in vivo populations, the substance may be delivered by administration to the host subject.

“Wnt activation” as used herein in connection with a substance or composition is an activation of the Wnt signaling pathway.

“Wnt activator” as used herein refers to a substance that activates the Wnt signaling pathway.

“CHIR99021” (often abbreviated herein as “C”) is a chemical compound having the chemical formula C₂₂H₁₈Cl₂N₈ and the following alternate names: CT 99021; 6-[[2-[[4-(2,4-dichlorophenyl)-5-(5-methyl-1H-imidazol-2-yl)-2-pyrimidinyl]amino]ethyl]amino]-3-pyridinecarbonitrile. Its chemical structure is as follows:

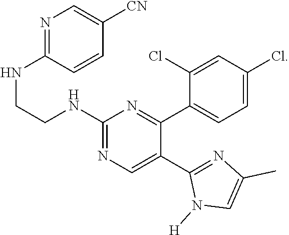 [SMILES: Cc1c[nH]c(-c2cnc(NCCNc3ccc(C#N)cn3)nc2-c2ccc(Cl)cc2Cl)n1]

DAPT (often abbreviated herein as “D”) is a chemical compound having the name N-[N-(3,5-Difluorophenacetyl)-L-alanyl]-S-phenylglycine t-butyl ester. Its chemical structure is as follows:

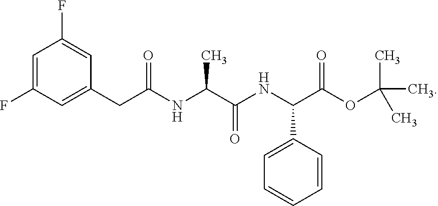 [SMILES: C[C@H](NC(=O)Cc1cc(F)cc(F)c1)C(=O)N[C@H](C(=O)OC(C)(C)C)c1ccccc1]

KPT-330 is a compound having the following structure:

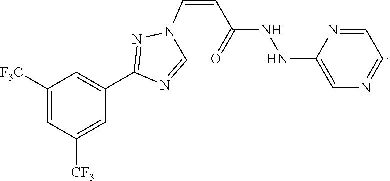 [SMILES: O=C(/C=C\n1cnc(-c2cc(C(F)(F)F)cc(C(F)(F)F)c2)n1)NNc1cnccn1]

KPT-8602 is a compound having the following structure:

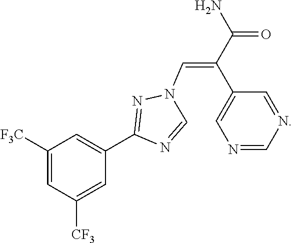 [SMILES: NC(=O)/C(=C/n1cnc(-c2cc(C(F)(F)F)cc(C(F)(F)F)c2)n1)c1cncnc1]

Leptomycin B is a compound having the following structure:

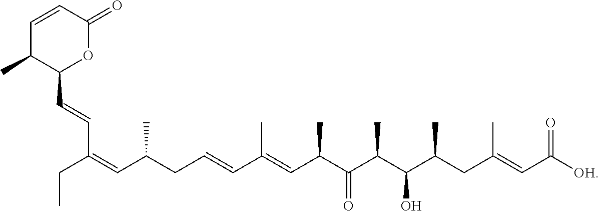 [SMILES: CCC(=C/[C@H](C)C/C=C/C(C)=C/[C@@H](C)C(=O)[C@@H](C)[C@H](O)[C@@H](C)C/C(C)=C/C(=O)O)/C=C/[C@@H]1OC(=O)C=C[C@@H]1C]

“Valproic acid” (VPA or “V”) has chemical formula C₈H₁₆O₂ and the following alternate name: 2-propylpentanoic acid. The sodium salt of valproic acid can also be used in place of VPA, and the term “VPA” is used interchangeably herein to refer to VPA or pharmaceutically acceptable salts thereof, such as the sodium salt. Its chemical structure is as follows:

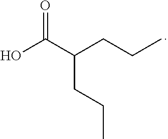

### DETAILED DESCRIPTION

A description of example embodiments follows.

The methods described herein can be used to differentiate leucine-rich repeat-containing G-protein coupled receptor 5-positive (LGR5⁺) intestinal cells. In general, the methods include contacting LGR5⁺ cells with an inhibitor of exportin 1 (XPO1). In some embodiments, the methods also include contacting the LGR5⁺ cells with a Wnt agonist, such as CHIR99021. In some embodiments, the methods also include contacting the LGR5⁺ cells with a Notch inhibitor, such as DAPT. In some embodiments, the methods include contacting the LGR5+ cells with an inhibitor of XPO1, a Wnt agonist, and a Notch inhibitor. Thus, in some embodiments, a Wnt agonist is not needed; in other embodiments, a Notch inhibitor is not needed; in yet other embodiments, neither a Wnt agonist nor a Notch inhibitor is needed. The cells are differentiated to mature intestinal cells (e.g., Paneth cells), which can be identified as CD24-mid/LYZ+.

While experiments described herein were performed in mice, relevant pathways and cell markers share a large degree of homology between mice and other mammals, such as humans. Thus, methods and principles that are demonstrated in mice are applicable to other mammals, such as humans.

### INTRODUCTION

The intestinal epithelium is a complex tissue that plays a key role in digestion and mediates important innate and adaptive immune functions. The small intestinal epithelium is formed by a single layer of cells arranged into villi—primarily composed of enterocytes, absorptive cells, and secretory Goblet cells—and crypts, which contain intestinal stem cells (ISCs) and secretory Paneth cells (PCs). Cells located in the villi are specialized for nutrient absorption, cells residing in the crypts are integral to regenerating the intestinal epithelium, and specialized cells throughout the epithelium provide for a protective barrier between host and microbe. Goblet cells secrete mucins into the lumen of the intestine to create a physical barrier between the host and the bacteria populating the gut. PCs contribute to the barrier by secreting antimicrobial proteins (AMPs) to form a biochemical barrier. In a healthy small intestinal epithelium, PCs are potent modulators of the gut microflora through the known secretion of multiple antimicrobial protein families including lysozyme (LYZ), angiogenin, ribonuclease A family, regenerating islet-derived 3 gamma (REG3G), and peptides such as cystine-rich (CRS) peptides and alpha-defensins (DEFA). PCs also secrete cytokines including interleukin-17 (IL-17) and are involved in signaling across the innate and adaptive immune system. The gut microbiota participates in a variety of different functions including metabolism, host defense and immune development and has been linked to pathogenesis in gastrointestinal, autoimmune, and other diseases.

Genetic, morphological, and functional alternations in PCs have been shown to drive microbial dysbiosis, impaired intestinal epithelial barrier function, and inflammation. This includes the heterogeneous collection of pathologies that manifest as inflammatory bowel disease (IBD). Genetic associations linked to impaired PC function in IBD populations include abnormalities in NOD2 (innate immune activation), ATG16L1 (granule exocytosis), and XBP1 (ER stress response). The AMPs secreted by PCs also play a crucial role in protection against infection from enteric pathogens. Notably, in in vivo murine models, PC-depleted and AMP-deficient mice are more susceptible to bacterial translocation and inflammation. As well, in necrotizing enterocolitis (NEC), AMP secretion and PC number is altered corresponding with intestinal immaturity and dysbiosis. The immature epithelial barrier appears to be more sensitive to bacteria and bacterial translocation, leading to excessive inflammation and systemic infection. Furthermore, PC disruption in mice replicates human NEC pathology, suggesting that PCs may initiate NEC. PCs have also been implicated in Graft versus Host disease (GvHD), which occurs after an allogeneic stem cell transplant in which the donor T cells cause an inflammatory response in the host. Patients with GvHD also exhibit a loss in PC number, reduced expression of AMPs, and dysbiosis. Notably, Gram-negative bacteria become more prevalent and, when paired with impaired barrier function, can lead to severe sepsis.

Additionally, growing evidence implicates the gut microbiota in the development of metabolic syndrome, which precipitates cardiovascular disease, type 2 diabetes, and obesity, affecting nearly a third of Americans. Interestingly, PC abnormalities relating to ER stress response have been correlated with the very obese. Furthermore, increasing the population dynamics of certain ‘protective’ bacteria has been shown to mitigate a pro-obesity effect and metabolic syndrome-associated low-grade inflammation; this microbiota modulation may be done in the future through a PC axis.

The importance of impaired barrier function and dysregulation of the gut microbiota in the etiology of these diseases suggests that PCs present a promising therapeutic axis. This has already been demonstrated in GvHD. Treatment with R-spondin1 (R-Spo1), a potent WNT agonist, can elevate the secretion of alpha-defensins and restore the dysbiosis seen in mice with GvHD by stimulating ISCs to differentiate into PCs. However, while treatment with R-Spo1 illustrates the importance of PC regeneration, it faces many challenges in clinical translation to humans. R-Spo1 is shown to significantly increase crypt size and hyperactive WNT activation is implicated in precancerous hyperplasia and PC metaplasia. While the effects of R-Spo1 are inconclusive with respect to malignancy, WNT signaling must be carefully balanced to ensure homeostasis not priming for cancer. Other signaling pathways known to drive PC differentiation, including Notch signaling, face similar challenges. Activation of Notch signaling amplifies the proliferative progenitor population and promotes an absorptive cell lineage. Conversely, deactivation of Notch signaling amplifies differentiation to all secretory cell types and secretory cell hyperplasia. As these pathways affect multiple cell types in the intestinal epithelium and may lead to hyperplasia, they are not therapeutically viable. Therefore, a more specific PC-targeted treatment to accomplish PC regeneration is desirable.

### The Cellular Model Bottleneck in Drug Discovery

Cellular models have long been a backbone of drug discovery, serving as simplified but useful proxies of human biology and enabling fundamental discoveries in disease. Further, the proliferation of high throughput screening activities since the 1990s has only increased the value for simple, representative cellular models in drug discovery. However, many simple to use cell lines and models have a relatively large disconnect from human biology and disease physiology which is increasingly limiting in emerging therapeutic interests (including mining the gut microbiota) which require high-fidelity models of complex disease biology. For every agent brought to market, countless more identified in cell models have failed to provide therapeutic effect. These ‘misses’ can, in part, be attributed to failures of physiological representation in the existing models. The intestinal epithelium is one such example of biological complexity: it contains regenerative adult ISCs, as well as secretory Paneth, goblet, and enteroendocrine cells, which collectively mediate host-microbe communication and whole-body physiology, and are directly involved in myriad diseases. Efforts to improve the physiological-representation of intestinal models range from the use of tissue explants to organoid models and organ-on-a-chip approaches; however, these systems are often inflexible, poorly characterized and scaled, limiting their utility in early drug discovery.

Organoid models have proven useful for assessing the impact of genetic mutations on the overall tumorigenic capacity of ISCs and in cancer therapeutic screening. However, their application to polygenic inflammatory disease has been limited. While cancer-causing mutations present a clear proliferative phenotype in organoids, phenotypes not affecting proliferation, such as those in IBD, may not manifest if the correct cell state from in vivo is not accurately represented within the organoid. Yet, if the state can be reproduced in vitro, organoids offer an advantage in IBD therapeutic development, since the many loci identified through genome wide association study (GWAS) have been difficult to efficiently examine in vivo with animal models, and may not recapitulate human biology.

### Limitations to Scaling and Screening Organoid Cultures

Before organoid and organoid-derived cultures can be widely utilized in high-throughput screening (HTS) campaigns, there are multiple limiting aspects inherent to their method of culture which must be carefully addressed. Because organoids and organoid-derived cell clusters are conventionally grown suspended as a heterogeneous mix of cluster sizes and cell types in three dimensional (3-D) proteinaceous scaffolding (typically MATRIGEL®), it is difficult to reproducibly plate a uniform number of cells per well suspended in scaffold with automated liquid handling equipment. Plating by hand is out of the question due to the laborious and delicate plating process (as is typical of conventional organoid culture). Indeed, the plating of these cells is quite temperamental; in addition to cell clusters existing at a wide dispersion of sizes, they also rapidly sediment without continuous mixing and adhere to most non-protein coated tissue culture plastic ware. Furthermore, Matrigel is a thermo-responsive hydrogel, which will readily gel at room temperature and is highly viscous around its typical handling temperature of 4° C. Combined, these hurdles require serious adaptations and simplifications to the plating procedure to even attempt a scaled down (from 24- or 48-well plates to 96- and 384-well plates) and increased throughput approach.

Another area of appreciable difficulty is in developing and adapting scalable assays of organoid phenotype or behavior. Because organoid culture is inherently non-monolayer, non-confocal high-content imagers will not provide a consistent readout. Further gene-driven reporter assays may have variable presentation in heterogeneous cell populations of organoids, and heterogeneous cultures may further limit assay signal-to-noise in phenotypes beyond proliferation, where measuring cell type-specific phenotypic changes, such as shifts in PC function or even composition within the population is the ultimate goal. Considerations of the assay are equally important to those of plating procedure. As such, we set out to adapt the plating procedure such that we might be able to plate organoid-derived PCs in a fully-automated and high-reproducible fashion which is amenable to a simple, robust, and scalable measure of PC function.

### EXEMPLIFICATION

### Example 1: Scalable Screening of In Vitro Paneth Cells Informs Function, Development, and Survival

### Results

### Scalable Platform to Study Paneth Cell Development and Function

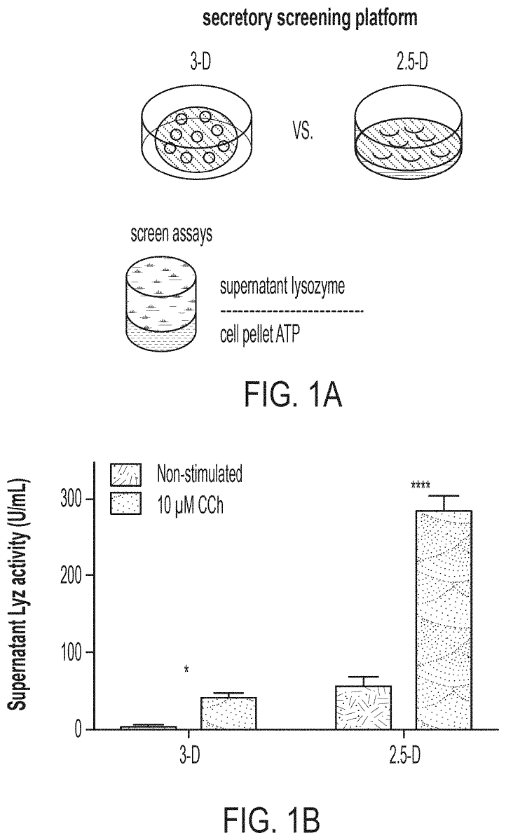

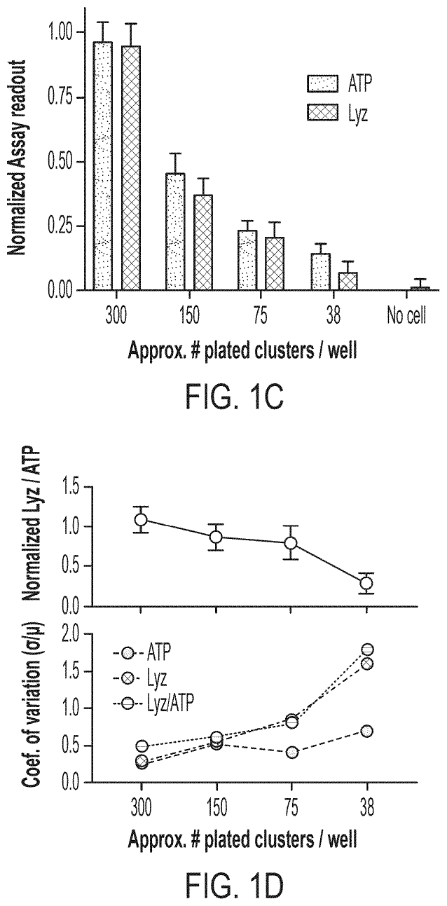

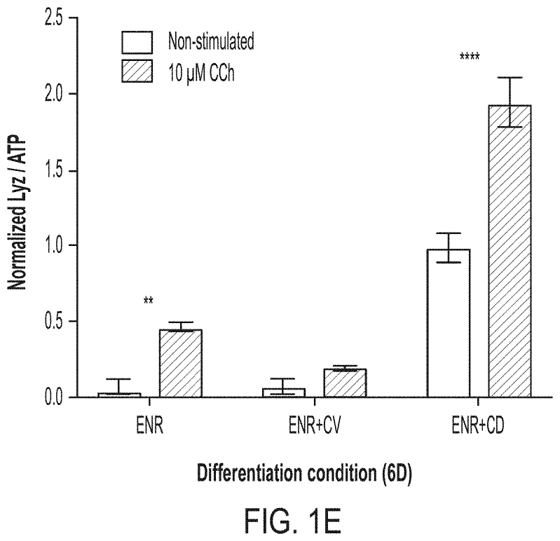

Building off our high-fidelity in vitro PC model (see 3), we set out to develop a scalable platform to assess PC function in vitro as a means to study the effects of host and microbe-derived agents, as well as small molecule drugs, on PC differentiation and function at scale. Starting from our assay of LYZ secretion into media supernatants as a measure of PC function and enrichment, we sought to develop a scalable, functional, phenotypic assay for screening ENR+CD-treated cells. To overcome the limitations to scaling organoid-derived cultures, we first sought to develop a method to preserve the important material and signaling cues supplied by Matrigel scaffolding while enabling automated plating through robotic liquid handlers used in high-throughput screening. We adapted the conventional “3-D” Matrigel droplet culture approach to a 96-well plate pseudo-monolayer “2.5-D” scheme in which organoids are re-plated partially embedded on the surface of a thick layer of Matrigel (at the Matrigel-media interface) rather than fully encapsulated in the Matrigel structure. This allows for a two-step automated plating procedure, where Matrigel is first deposited and gelled in 96- (or 384-) well plates, and culture media containing suspended cell clusters is then added into each well, and briefly centrifuged at low force to loosely deposit clusters on the surface of the thick Matrigel scaffold. This allows for Matrigel plating, cell seeding, and media additions to be fully automated by a liquid handler and readily scaled. Further, because the deposited cell clusters are now apically-exposed to the culture media, assaying apically-secreted agents (such as LYZ) should be greatly enhanced, while allowing for the multiplexed assaying of underlying embedded cells (FIG. 1A). Indeed, 6-day differentiated ENR+CD cells plated at equal density in 96-well plate in 2.5-D versus 3-D significantly yielded greater levels of both basal and induced secretion of LYZ into cell culture media following a 3-hour stimulation (FIG. 1B).

Next, we sought to determine the range over which our platform and paired assays would provide a near-linear signal measurement with manageable well-to-well variation, such that with proper controls we may forgo the use of standard curves and thereby increase useful sample space per plate. We seeded a range of 6-day ENR+CD differentiated clusters per well and assayed for cellular ATP as well as basal LYZ secretion over 3 hours. Between ˜300 clusters /well (˜10 clusters/μL media) to ˜38 clusters/well (˜1 cluster/μL media) we see a near linear trend in both adherent cells (ATP) and basal LYZ secretion (FIG. 1C), suggesting a wide working range. Further, we see down to ˜75 clusters/well a similar ratio of basal LYZ/ATP, suggesting that over the range 300-75 clusters/well the cells are phenotypically similar, additionally the range 300-75 clusters/well provides for the lowest coefficient of variation (a measure of well-to-well reproducibility) (FIG. 1D). As such, we settled on the optimal plating density as between 300-75 clusters/well (10-2.5 clusters/μL media). Using this optimal density and assay format we then demonstrated that our 2.5-D platform in a 96-well plate provided an ability to discriminate PC function between conventional, ISC-enriched, and PC-enriched organoids (FIG. 1E).

With our screening platform established, we next sought to apply it as a tool to investigate and validate the actions of proposed agents which modulate in vivo PC function. To accomplish this, we set out to test both the short-acting (over three hours) and long-acting (over 48 hours) potentiation of multiple agents which have been previously reported to alter the antimicrobial secretion or functional capacity of PCs, including the bacterial derived E. coli Lipopolysaccharide (LPS) and gram-positive muramyl dipeptide (MDP), and host-derived cholinergic agonism (carbamyl choline—CCh), and cytokines interferon-gamma (IFN-γ) and tumor necrosis factor alpha (TNF-α).

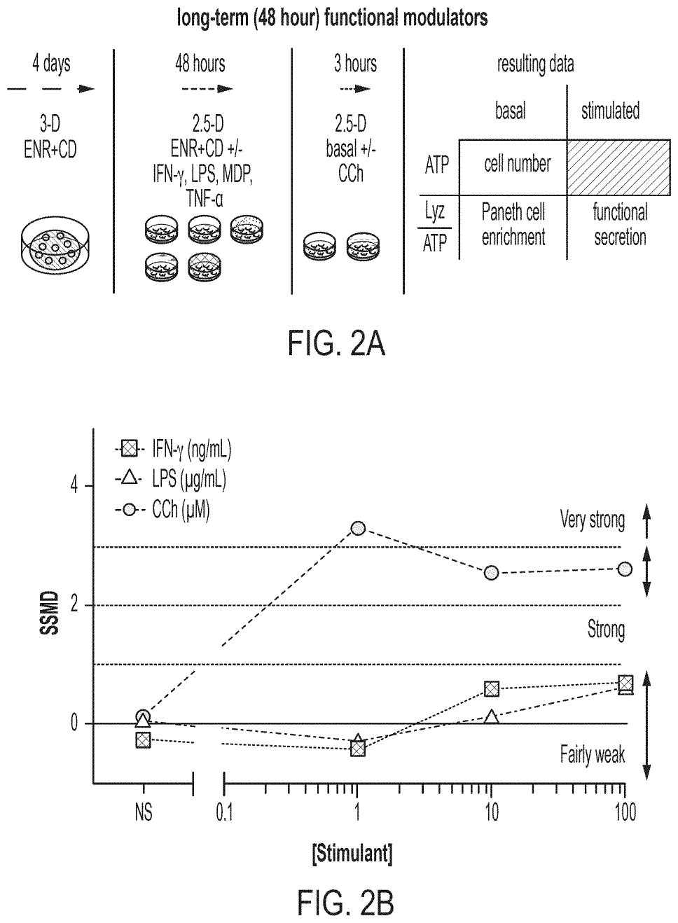

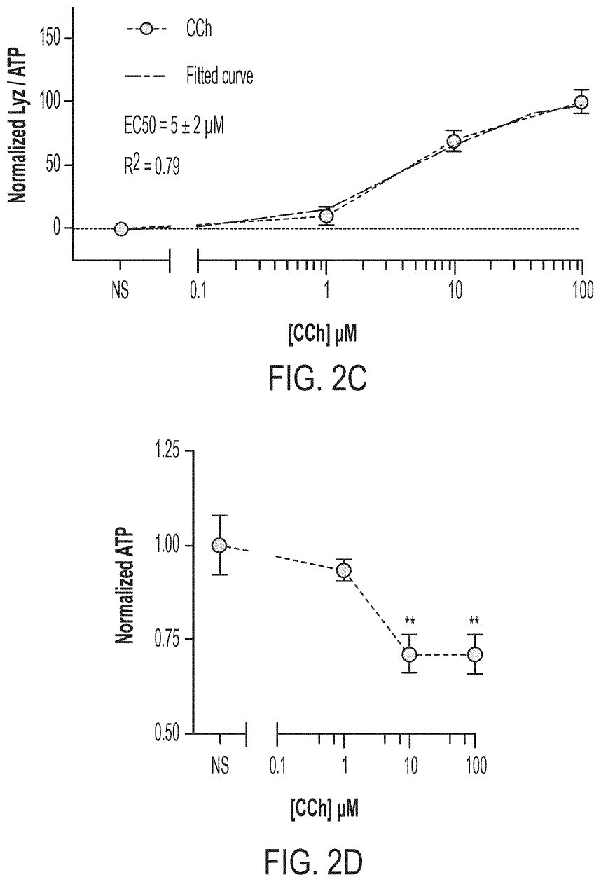

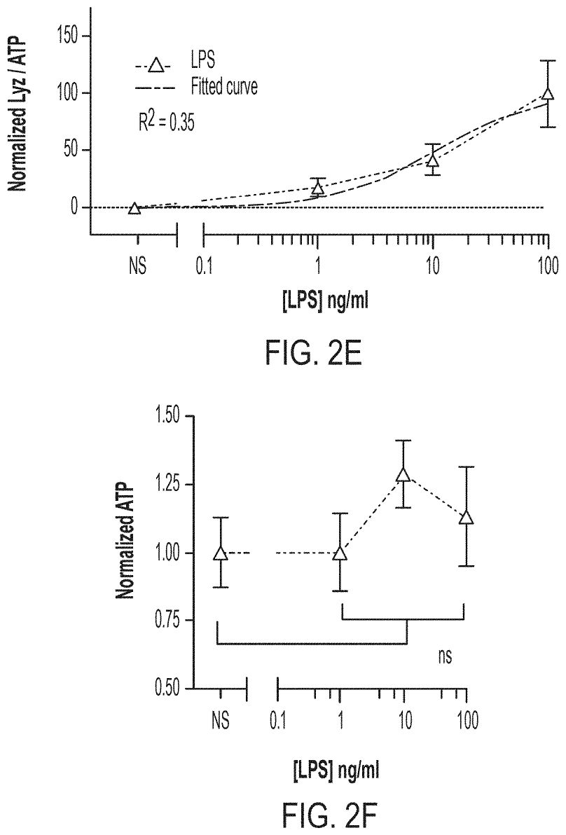

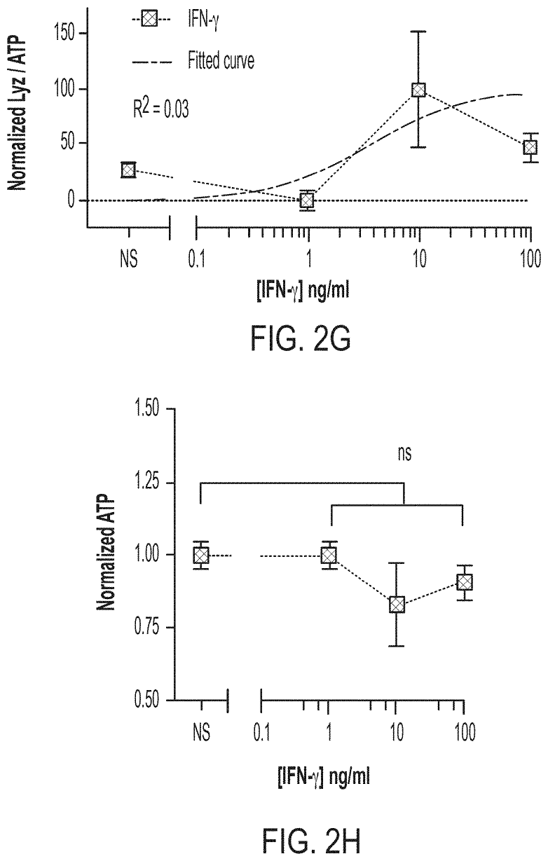

We first sought to profile the potential short-term actors, IFN-γ, LPS, and CCh, and see if with the added resolution of this first-of-its-kind PC platform, we might be able to determine high-resolution EC50's. To generate sufficient cells for plating out all necessary conditions we first expanded and enriched ISCs in traditional 3-D culture and subsequently differentiated for 4 days towards PCs (ENR+CD). Next, we passaged and plated these cells into 96-well plates as 2.5-D and continued differentiating for an additional 2 days in ENR+CD. On day 6 of differentiation, each well was then washed with basal media repeatedly and stimulated with its respective agent, with a measurement of supernatant LYZ paired to cell pellet ATP (FIG. 2A). This screen was repeated on three separate occasions with cells from three distinct murine donors and analyzed combined. We first wanted to determine the approximate standardized effect size of each treatment at each dose, which was accomplished through the replicate-based strictly standardized mean difference (SSMD), an effect size measure based on the statistics of contrast variables, and particularly useful in screening applications as it is a measure which takes into account both mean difference and variance in a single measure. Using the replicate based UMVUE SSMD (see Materials and Methods, High-throughput screening: 96-well format), we identified that only CCh was a strong effect short-term modulator of LYZ secretion, with both LPS and IFN-γ having fairly weak effect sizes across all tested doses (FIG. 2B). Indeed, we see that the addition of CCh leads to a clear dose-response effect, which is well-fitted by a least-squares fit (R²=0.79) and provides an expected EC50 of 5+/−2 μM, while at higher doses also having a significant effect on cellular ATP (adj. p<0.01 for 10 and 100 μM doses) (FIGS. 2C and 2D). Additionally, we see that the fairly weak effects of both LPS and IFN-γ manifest in poor dose-response fits (LPS: R²=0.35, IFN-γ R²=0.03), and no significant changes in cellular ATP (FIGS. 2E-H). However, observing the LPS curve suggests that the concentration range tested may be sub-optimal, and may warrant future study at higher doses or with LPS derived from alternative bacterial species. In total, we see that our 2.5-D platform does provide a reasonable system for assessing and screening for potential short-term modulators of PC antimicrobial secretion. We demonstrate the ability to both distinguish strong and weak effect agonists of LYZ secretion, and in the case of strong agonists (in this case CCh), are able to provide a relatively high-resolution dose-response profile with associated EC50 concentration.

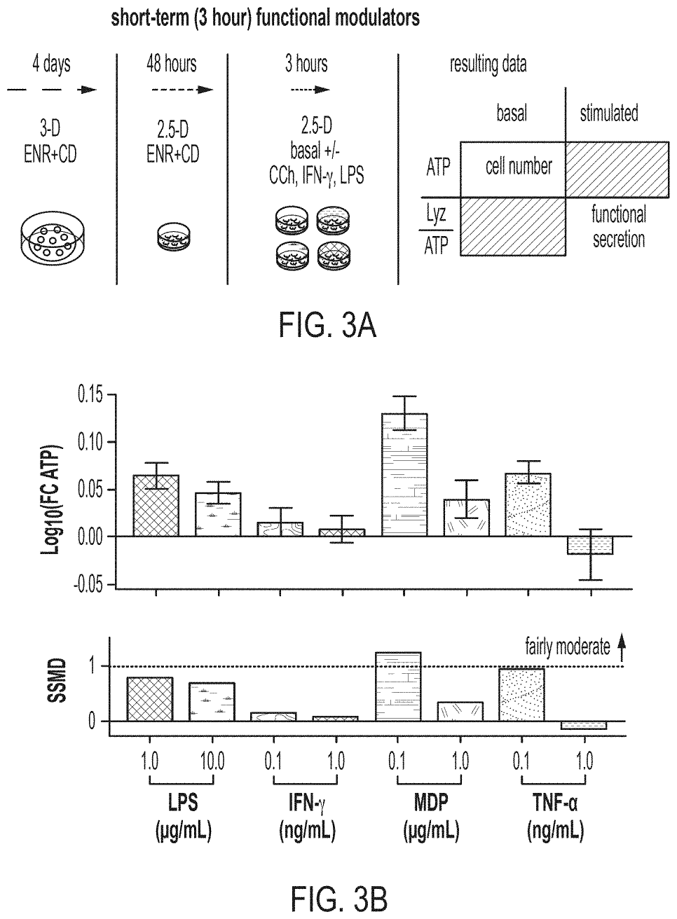

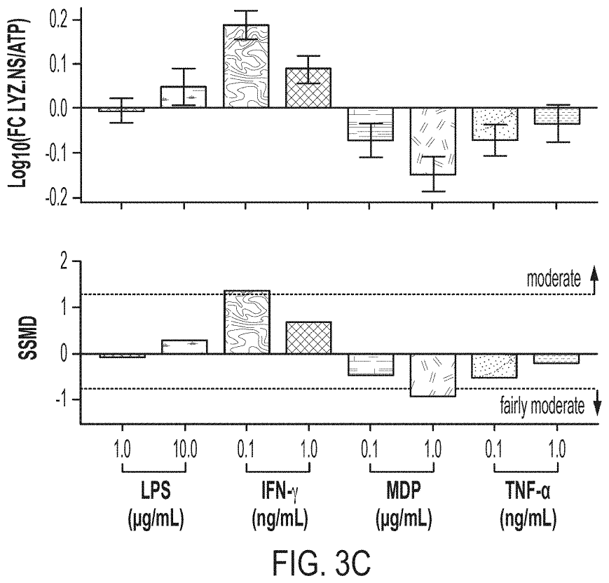

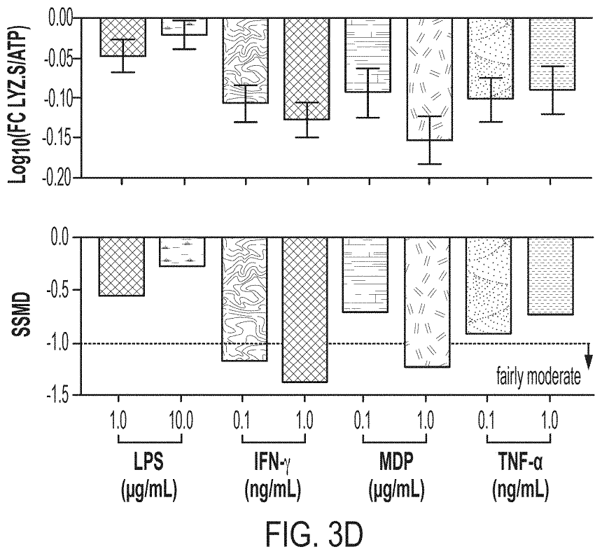

We next sought to profile potential long-term modulators of differentiated PC survival and antimicrobial secretion, as well as gauge the interaction between long-term modulators and short-term induced secretion (via CCh). To accomplish this, we employed a similar set up as in the short-term experiment, with the exception that upon plating day 4 ENR+CD cells we additionally supplemented in either MDP, LPS, TNF-α, or IFN-γ for the additional 2 days in culture and assayed all conditions for both basal and CCh-induced LYZ secretion over 3 hours on day 6 of differentiation (FIG. 3A). We see that the low doses of LPS, MDP, and TNF-α all show apparent increases in cell number, with the SSMD effect size of MDP as qualifying as fairly moderate and the remainder as weak (FIG. 3B). However, this increase in cell number does not translate to increases in basal LYZ secretion, instead we see that IFN-γ drives a moderate increase in secretion while MDP shows a fairly moderate decrease in basal secretion (FIG. 3C). Additionally, we see that the long-term addition reduces CCh-induced secretory function relative to the untreated control for all agents (FIG. 3D). These effects are fairly moderate for IFN-γ, and MDP, and weak for LPS and TNF-α. This suggest two separate phenomena occurring for the addition of IFN-γ and MDP, with relatively little robust effect of TNF-α and LPS in this system. IFN-γ appears to be acting as a long-term inducer of antimicrobial secretion, which blocks the action of cholinergic agonists, while MDP appears to cause an expansion of non-PC cells within the system which possess a reduced secretory capacity.

In total, these results demonstrate a platform capable of scaling an organoid-derived culture reproducibly with a simple phenotypic assay for PC function (LYZ secretion). With this platform, an ability to assess potential short and long-term modulators of differentiated cell function was demonstrated. The particular in vitro PC model was validated by confirming the potent-secretory induction from cholinergic agonists (CCh) and the long-term secretory induction from the inflammatory cytokine IFN-γ, both previously reported phenomena. A primary strength of this PC screening platform is in studying agents that may enhance the pace of PC development and may serve as therapeutic candidates to increase or improve PC quality in diseases where there is a loss of PC number or function, such as ileal Crohn's disease.

### Primary Small Molecule Screen for Molecular Targets to Enhance Paneth Cell Development

While our scaled screening approach enables phenotypic study of differentiated PCs at scale, it also offers an opportunity to examine modulators of the differentiation ‘trajectory’ from ISC precursors. Intervening during the 6-day differentiation of ENR+CD organoids allows for the study of unappreciated molecular pathway activity which influences PC differentiation and may be readily translated beyond our model. We therefore sought to use our scalable “2.5-D” platform to demonstrate a proof-of-concept screen for developmental process of PCs in vitro, and to elucidate molecular pathways and small molecule agents which may afford an axis to enhance PC number therapeutically.

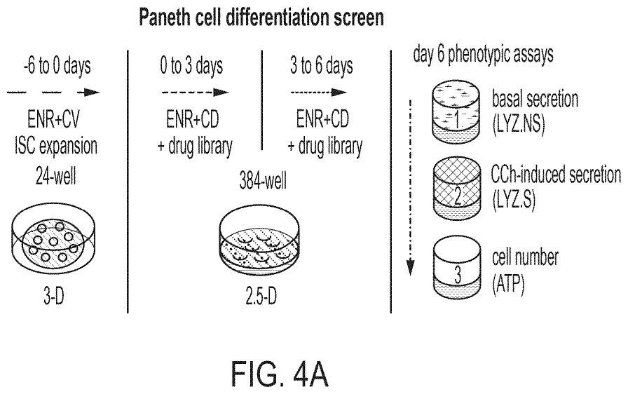

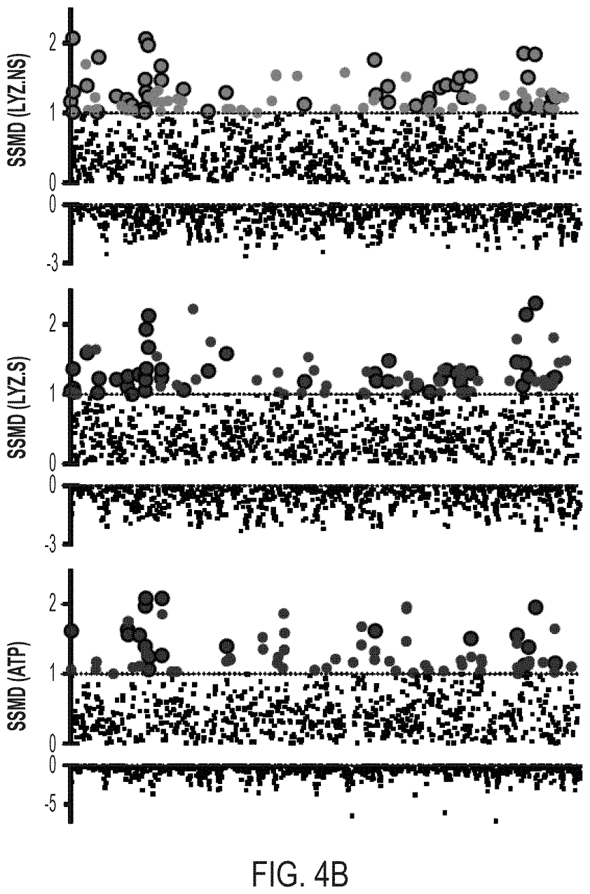

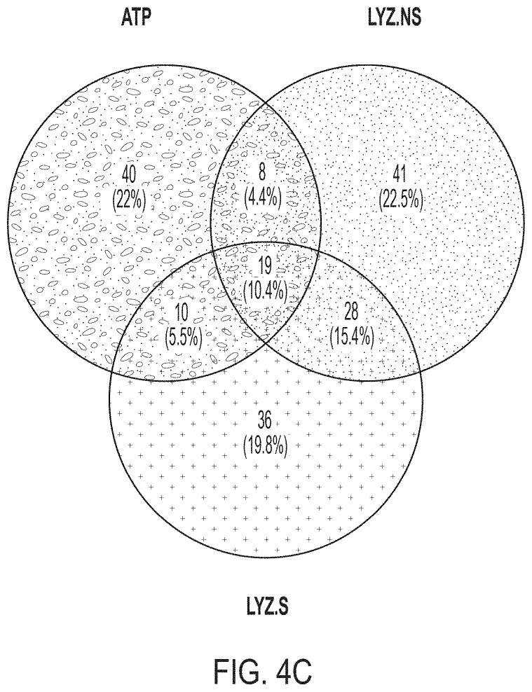

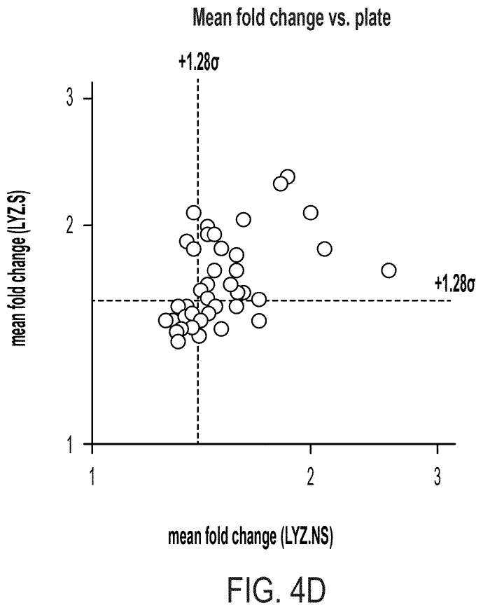

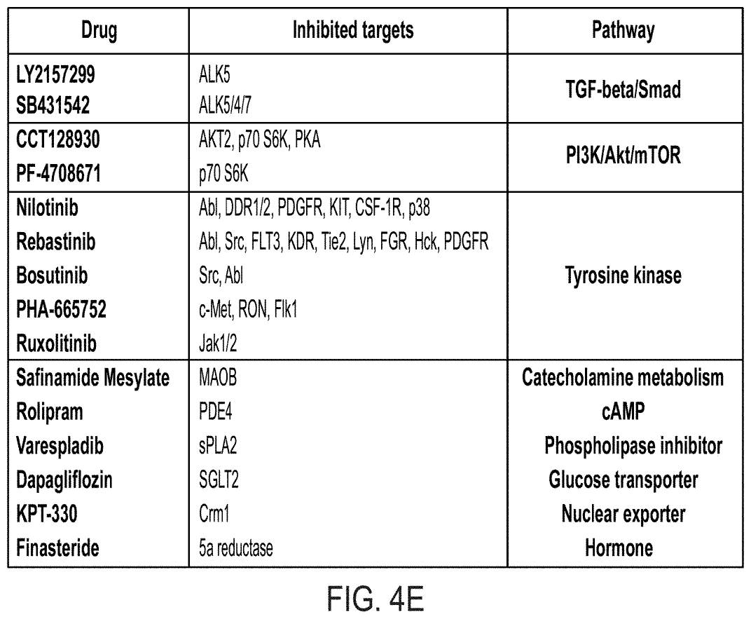

Using a modified version of our 96-well “2.5-D” system and functional assays, we screened for agents which enhance in vitro PC differentiation or survival using a target-selective inhibitor (TSI) library (Selleck Chemicals (Houston, Tex.) L3500) containing 433 small molecules covering 184 well-characterized unique molecular targets with high specificity. This library offers translational advantages as many of the molecules are either presently used in the clinic for a wide variety of conditions or have been used in clinical trials and animal models. We scaled our 96-well 2.5D system to a 384-well plate format with a single-well stimulation protocol and assessed the activity of secreted LYZ (basal—LYZ.NS and CCh-stimulated—LYZ.S) and cell pellet ATP following treatments of each compound. ISC-enriched “small clusters” from 3 biological donors were seeded as 3 replicate screens into differentiation media (ENR+CD), and then screened with each of the 433 different compounds at 4 doses covering the nano-molar to micro-molar range. By screening in the presence of the PC differentiation media we sought to assess how the library compounds may act outside of the known WNT and Notch pathways to influence PC differentiation or function, while simultaneously generating a PC-enriched system with which to robustly assay for PC function. We performed the three sequential assays 6 days after initial plating with ENR+CD+library treatment and had an additional media change and drug treatment at day 3 (see Materials and Methods, High-throughput screening: 384-well plating and assays) (FIG. 4A). Each screen plate was log₁₀ transformed, and LOESS normalized to reduce plate effects, and each well value was reported as fold change (FC), relative to the median assay value of its respective plate (under the assumption that many of the compounds and doses on the plate will not be biologically active and therefore serve as a suitable control).

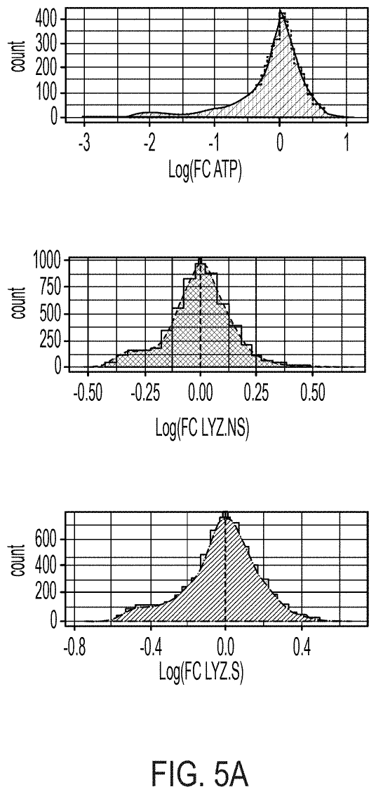

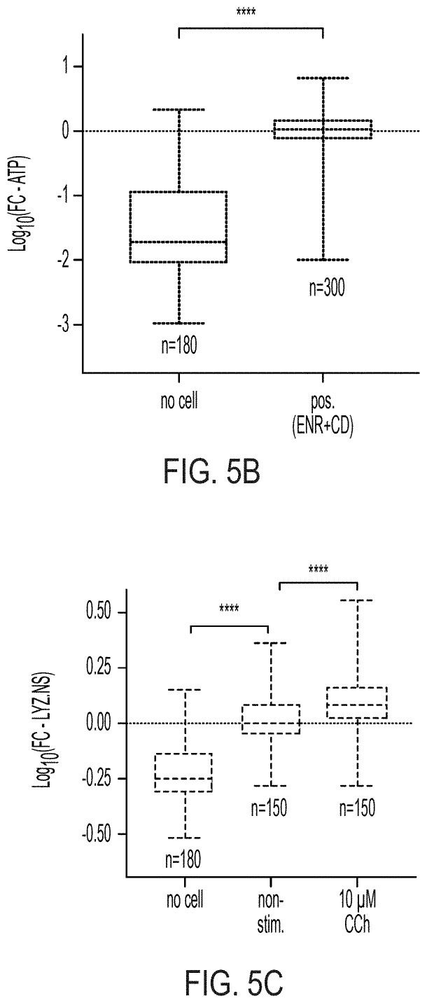

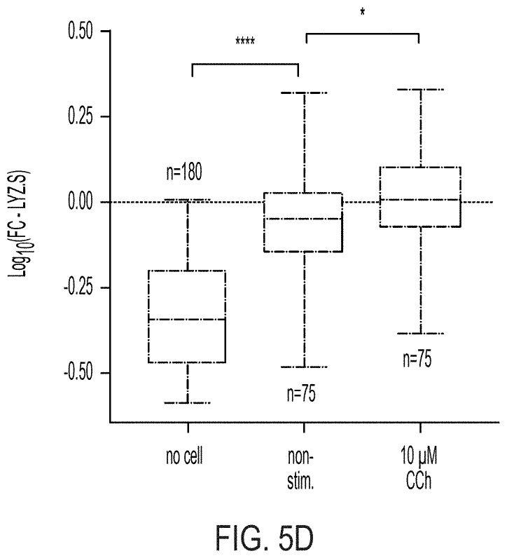

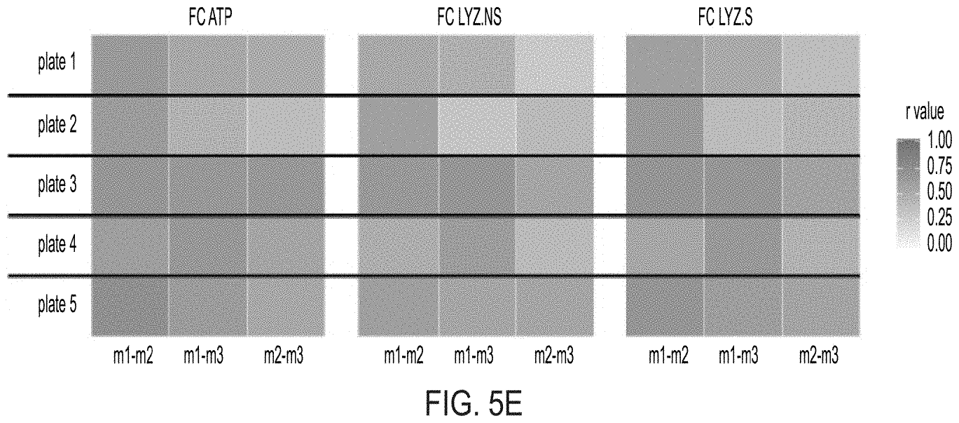

Prior to assessing individual treatment effect size, each assay was individually assessed for quality based on no cell and untreated (ENR+CD) controls following data normalization. Each assay was centrally-distributed similar to a normal distribution with slight low-end skew representing the included ‘no cell’ controls (FIG. 5A). As well, FCs of no cell controls versus untreated ENR+CD control wells were well-distinguished (p<0.0001), indicating untreated wells on average contained viable cells at screen conclusion (FIG. 5B). Further, in both LYZ.NS and LYZ.S assays untreated (ENR+CD) wells were assayed for basal and CCh-stimulated LYZ secretion as a measure of intended biological activity of plated cells at screen conclusion. In the LYZ.NS (first assay—all +drug wells are sampled for basal secretion) non-stimulated ENR+CD controls were significantly greater than that of no cell controls (adj. p<0.0001), and 10 μM CCh-stimulated ENR+CD controls was significantly greater than that of non-stimulated positive controls (adj. p<0.0001) (FIG. 5C). In the LYZ.S (second assay—all +drug wells are sampled for CCh-induced secretion) non-stimulated ENR+CD controls subsequently stimulated with 10 μM CCh versus non-stimulated (adj. p<0.05) and those doubly non-stimulated positive controls versus no cell controls (adj. p<0.0001) showed significant differences (FIG. 5D). Further, each plate across each replicate was relatively well-correlated for all three assays (FIG. 5E).

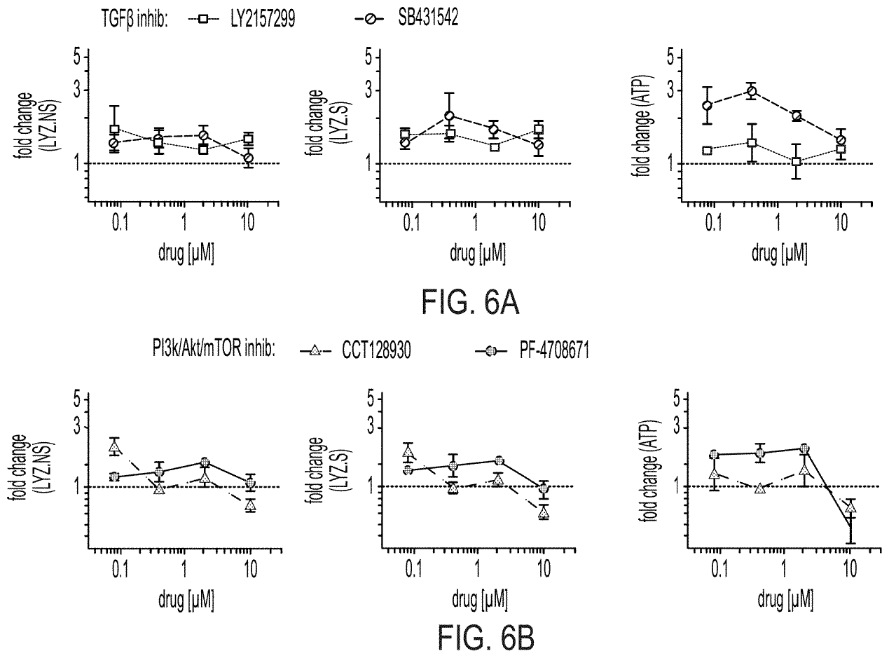

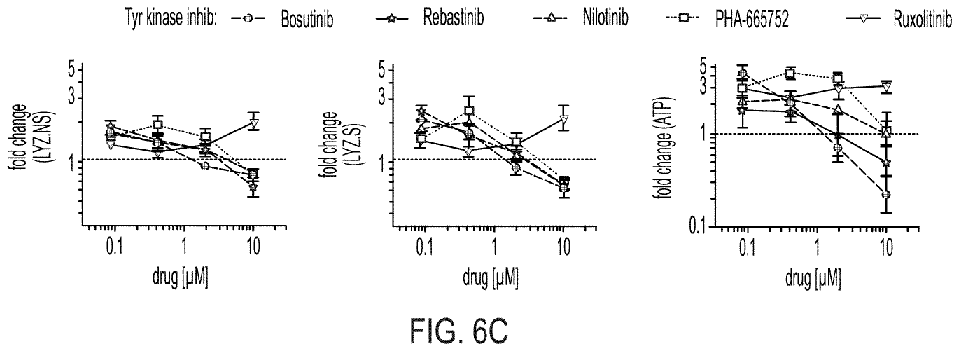

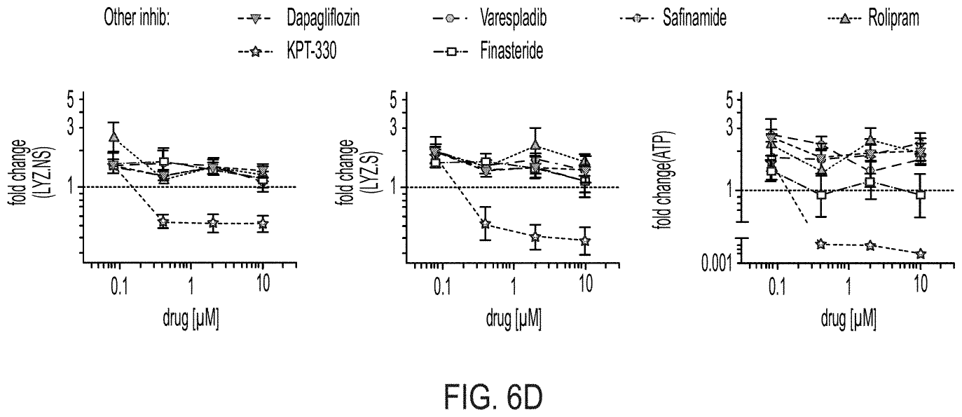

To assess treatment effect size and define primary screen ‘hits’, replicate SSMD was calculated (see Materials and Methods, High-throughput screening: 384-well primary screen format and analysis) and hits were determined by an SSMD greater than the false-positive and false-negative derived cutoffs (errors equalized to minimum for 3 replicates alpha=0.087; see Materials and Methods, High-throughput screening: 384-well primary screen format and analysis) (β_{α}_{₁}=0.997) for both LYZ.NS and LYZ.S assays (FIG. 4B). Of these 47 hits, 19 were also hits in the ATP assay, with the remaining 28 as hits in only both LYZ assays. Interestingly, a plurality of hits was assay-specific, suggesting that single-assay hits may either arise from system ‘noise’ or may suggest unique biological effects (FIG. 4C). We further refined the set of 47 double-LYZ assay hits by only including the most biologically ‘potent’ treatments, using a cutoff corresponding to treatments which would fall in the top 10% of a normal distribution (z-scored FCs>1.282) (FIG. 4D). 15 small molecules covering 9 unique reported molecular targets were thusly considered to be both biologically potent and statistically significant hits. When grouped by molecular target, most small molecules show relatively consistent dose-response effects, and for the most part show potency across a wide range of concentrations, with optimal efficacy in the low micromolar to high nanomolar doses (FIGS. 6A-D). For molecular targets with more than one hit treatment-dose of the same molecular target—TGF-β ALK5 inhibitors, and tyrosine kinase Abl inhibitors—only the strongest LYZ assay-performing treatment-dose was selected for further investigation (SB431542 and Nilotinib). As such, 13 small molecules were advanced to confirmatory ‘secondary’ screens to better profile their biological activity and rule out agents which may show inconsistent potency or excessive toxicity in a broader model of the intestinal epithelium.

### Secondary Screening Validates Potent Targets to Selectively Enhance Paneth Cell Differentiation In Vitro

To validate the 13 most potent enhancers of PC differentiation identified in primary screens, we conducted secondary screening using the same assays and screening format as before with an increased number of well replicates per treatment, a lower tolerance for false positives (without consideration for false negatives—see Materials and Methods, High-throughput screening: 384-well secondary screen format and early vs. full treatment analysis), and a more stringent control population (ENR+CD or ENR compared to whole plate in primary screen). Screening was performed with the identified 13 primary screen hits at a narrowed dose range—2-fold above and 4-fold below the most potent doses identified in primary screens, at 2-fold increments.

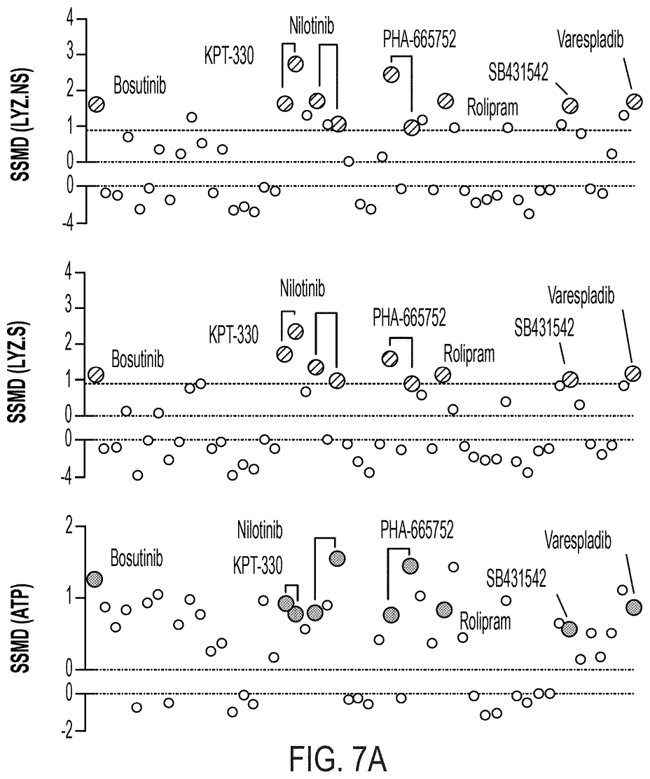

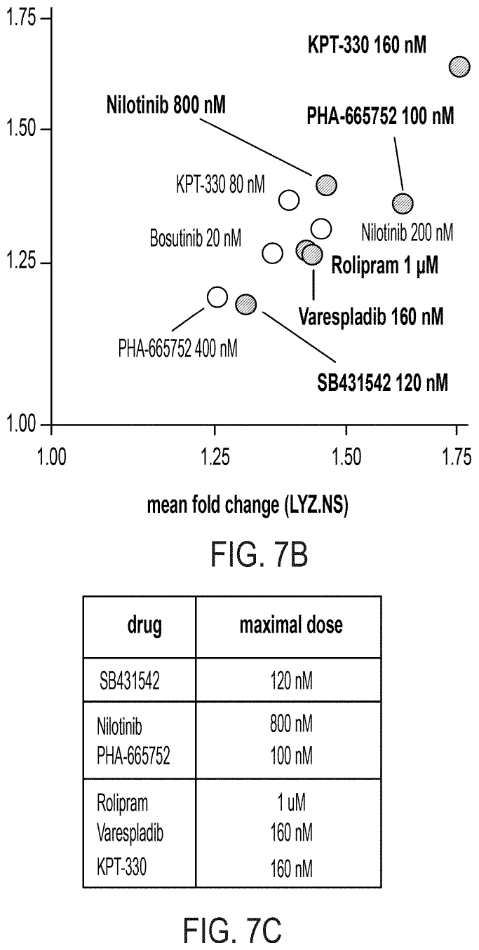

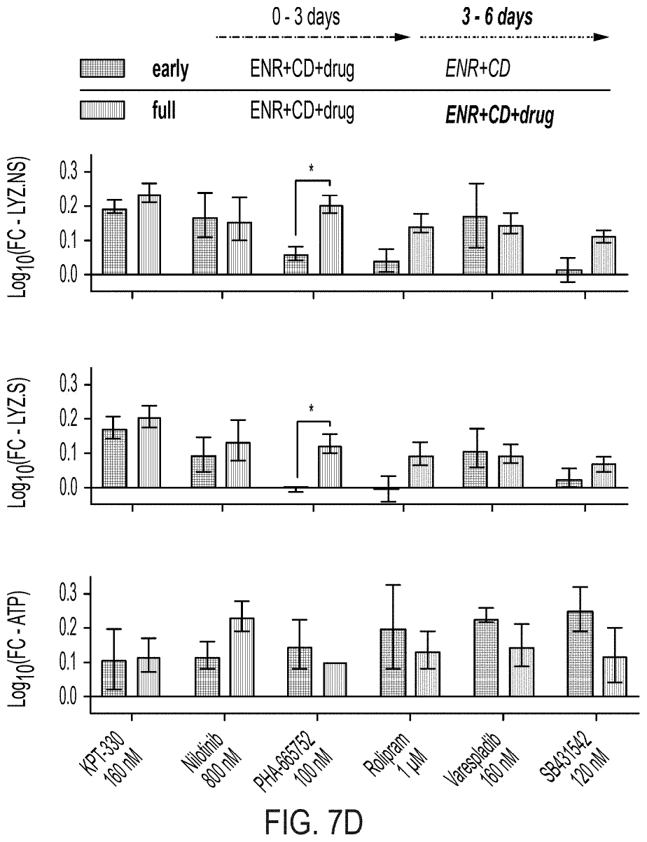

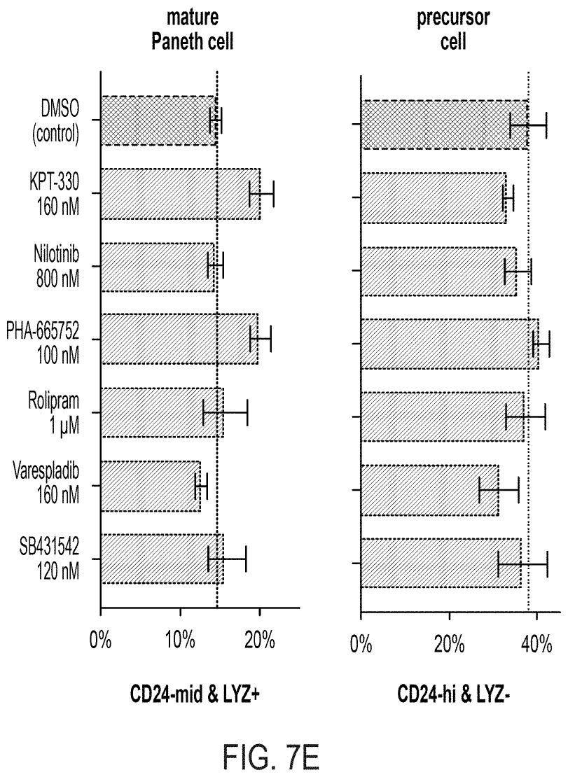

We again assessed treatment effect size with replicate SSMD (using a difference between well replicates and median assay value for all same-plate control wells—see Materials and Methods, High-throughput screening: 384-well secondary screen format and early vs. full treatment analysis) and hits were considered statistically validated by an SSMD greater than the false-positive-derived cutoffs (β_{α}_{₁}=0.89 for 8 replicates with alpha=0.05; see Materials and Methods, High-throughput screening: 384-well secondary screen format and early vs. full treatment analysis) for both LYZ.NS and LYZ.S assays (FIG. 7A). Per this new cutoff, 10 dose-treatment combinations corresponding to 7 small molecules were chosen as hits, with every passing dose-treatment having a greater than 0 ATP effect size. We also profiled the biological potency (mean fold change between treatment and ENR+CD+DMSO for the LYZ.NS and LYZ.S assays), of the 10 validated dose-treatment combinations, showing that the compounds increased basal and stimulated LYZ secretion by 25%-75% relative to the control (FIG. 7B). For each compound with multiple validated doses (KPT-330, PHA-665752, and Nilotinib), the most biologically active dose was advanced, and, because Nilotinib and Bosutinib have similar known mechanisms, only Nilotinib (the more biologically potent) was advanced (FIG. 7C) to additional profiling.

With these 6 validated small molecules, we next wanted to begin understanding how they might be acting to enhance PC function during the course of a 6-day ENR+CD differentiation. We reasoned that if these molecules were acting as enhancers of differentiation, rather than boosting terminal function in our secreted LYZ assay, they should show similar increases in function when treated during the first 3-days of differentiation (early) versus a full 6-day treatment (FIG. 7D). Indeed, we find this to be the case for every drug tested but one, PHA-665752, which shows a significantly (adj. p<0.05) lower improvement in function with early treatment versus full in both LYZ.NS and LYZ.S assays. None of the tested compounds had significant differences in ATP (a measure of cell number). It is worth noting we also observe a similar lower (non-significant) functional improvement in both LYZ assays for Rolipram and SB431542.

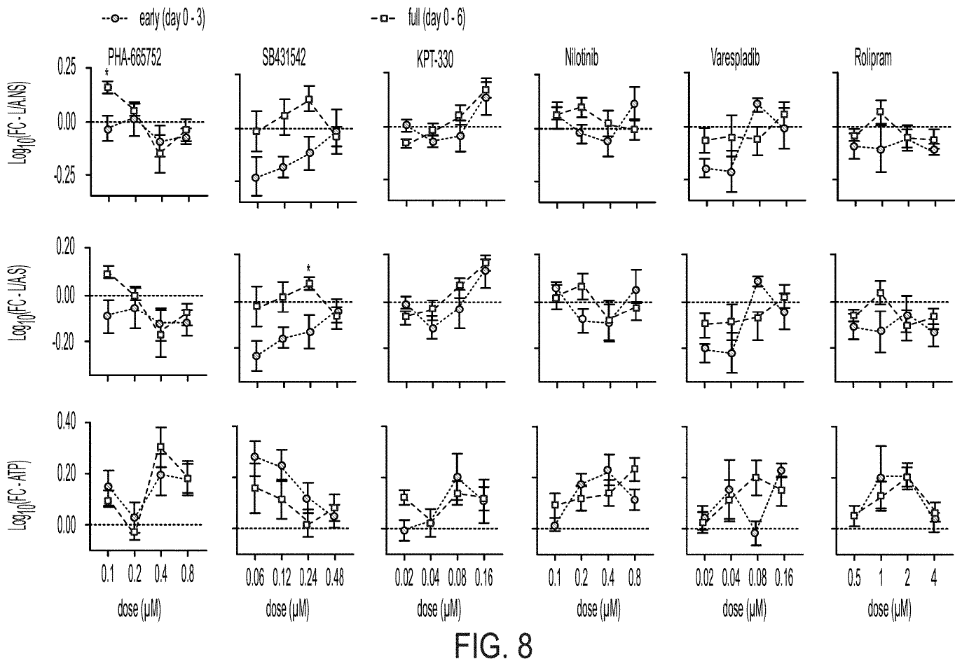

To further clarify these differences between early and full treatment during differentiation, we looked at the differences between early and late treatment across a range of doses for the 6 compounds in the LYZ assays normalized to their matched ATP values (a measure of LYZ/ATP suggests functional capacity per cell) as well as their ATP basis. In this case we again see that at the lowest (and maximal) dose for PHA-665752 (100 nM), there is a significant difference (adj. p<0.05) between early and full for LYZ.NS/ATP and a suggestive trend for LYZ.S/ATP with no difference in ATP alone (FIG. 8).

Further, we see that for SB431542, there is a significant (adj. p<0.05) difference between early and full treatment at the 240 nM dose in the LYZ.NS/ATP assay and a suggestive trend across all doses for both ATP-normalized LYZ assays between early and full treatments. Finally, we do not see a trend or significant difference between early and full treatments for Rolipram or the 3 other validated small molecules. In total, this suggests that both PHA-665752 and SB431542 may be acting to alter the functional capacity of differentiating cells rather than (or in addition to) driving early progenitor differentiation, while the remaining four compounds appear to be primarily acting through an early, progenitor-directing effect.

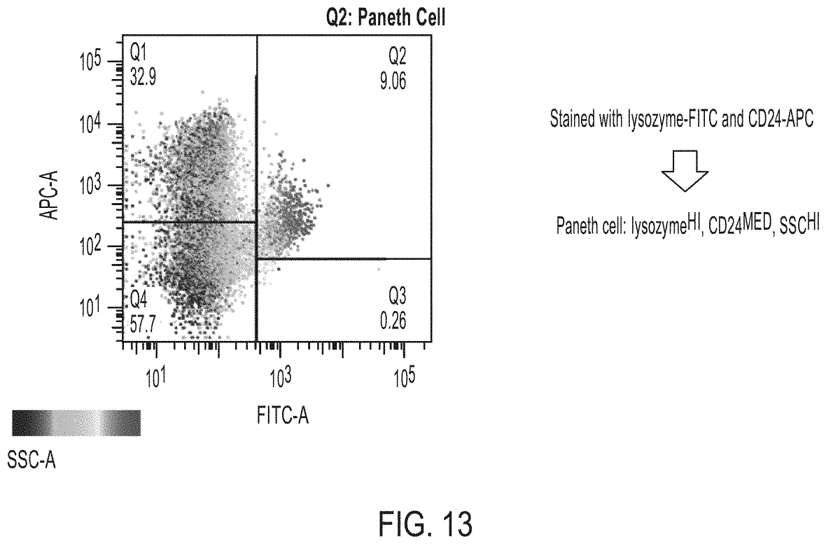

As further proof of how these molecules may be altering the differentiation trajectories of PC and secretory progenitor populations we performed flow cytometry profiling to identify changes in mature PC (CD24-mid & LYZ+) and secretory precursor (CD24-hi) populations (see gating in FIG. 13). Cells processed for flow cytometry were first gated to exclude potential debris or doublets (via widely accepted forward and side scatter gating), and subsequently gated for viability (via Zombie-violet viability dye exclusion), all viable cells were considered for CD24 & LYZ expression analysis. Following a 6-day differentiation in ENR+CD+treatment, all of the 6 small molecules result in increased mature PC population relative to control, however, only treatment with KPT-330 provided a statistically significant increase (adj. p<0.01) (FIG. 7E). Further we see no significant changes in the precursor populations, but a trend to a smaller population following KPT-330 treatment, and a higher proportion with Nilotinib. In total, these results suggest that the addition of these 6 small molecules is likely enhancing the conversion of non-secretory precursors to secretory precursor populations, which then have similar conversion to mature PCs after 6 days of differentiation.

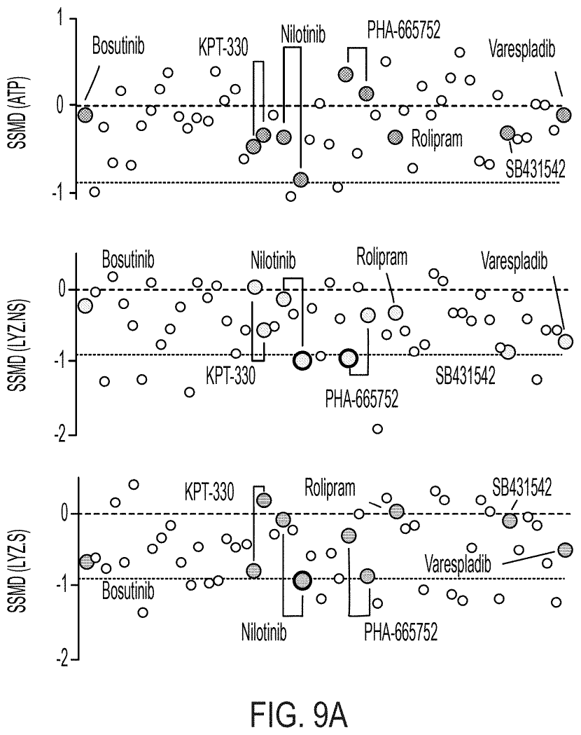

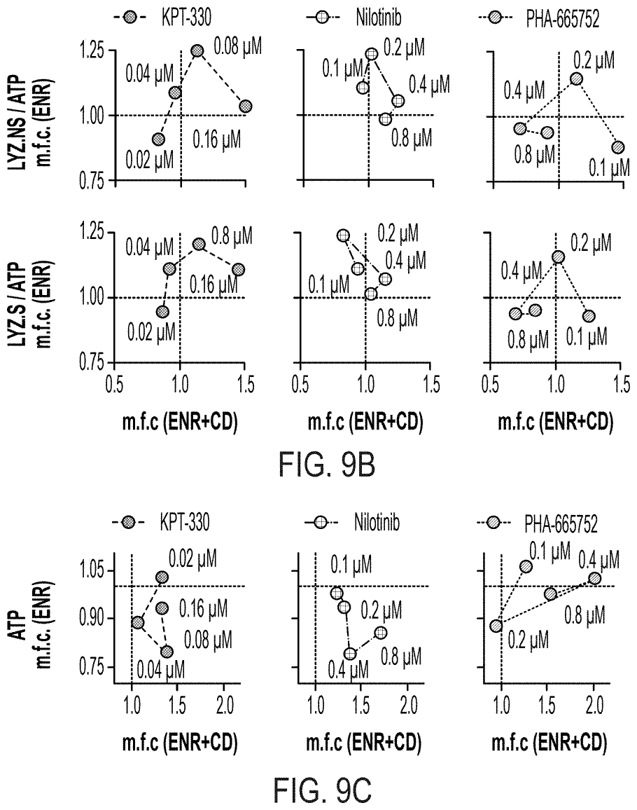

Finally, we sought to profile the effects of the 13 small molecules identified in primary screening, and in particular the 6 validated small molecules in the conventional organoid (ENR), to 1) assess any toxicity of these small molecules on the other epithelial cell populations and 2) assess the potential for these small molecules to drive appreciable PC differentiation in the absence of strong WNT agonism and Notch inhibition (ENR+CD). Here we again used the same screen format as previously, with a 6-day differentiation and day 3 media change and re-treatment in an ENR-supplemented media. We again calculated a replicate SSMD based on the differences between ENR+DMSO and ENR+treatment conditions from 8 well replicates. However, because toxicity is of primary interest in this instance, we implemented a cutoff based on the negative effect of each treatment on well ATP (−0.89), as well as secondary negative effect cutoffs for each LYZ assay. We see that for each compound that was validated in secondary ENR+CD screening, there is no significant decrease in ATP, however, single treatments for both Nilotinib and PHA-665752 have significantly lower LYZ.NS (PHA-665752) and LYZ.S (Nilotinib) readings (FIG. 9A). This suggest that none of our 6 validated molecules present a particularly toxic effect on cells within a conventional organoid, but that for Nilotinib and PHA-665752, there may be an effect which reduces the (already low) abundance of PC-like cells.

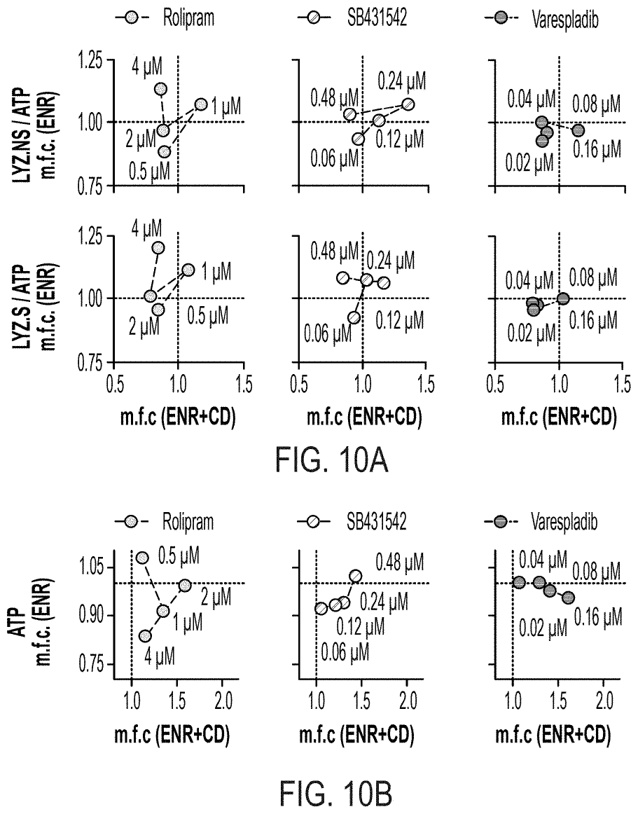

We also began an assessment of how our 6 validated molecules change PC content or quality in the presence and absence of WNT agonism and Notch inhibition (+/−CD), by profiling the mean fold change in LYZ.NS/ATP and LYZ.S/ATP, as well as baseline ATP, compared between the 6-day conventional (ENR) organoid differentiation and the ENR+CD differentiation. We see that for the strongest inducer of PC differentiation in ENR+CD, KPT-330, there are reasonable increases in LYZ secretion per cell in ENR as well, suggesting that KPT-330's mechanism of differentiation may be independent of the +CD induction (FIGS. 9B and 9C). For the five other small molecules, we do see doses between both systems which have positive mean fold changes, with the exception of Varespladib, which has minimal effects in the ENR-only system (FIGS. 9B, 9C, 10A, and 10B). In total, this suggests that, to the extent detectable by these LYZ secretion assays and with the exception of Varespladib, the 6 small molecules identified from this organoid-derived PC-differentiation screening platform are able to potentially independently drive PC differentiation or provide functional improvements.

### DISCUSSION

We sought to develop a scalable platform for the reproducible measurement of in vitro PC development and function, and with this platform identify novel agents to enhance in vivo PC differentiation and function. We identified a set of small molecules that likely act on a range of molecular targets to robustly increase PC differentiation through means distinct from WNT and Notch signaling. The reported mechanisms of action of these agents in other cell and in vivo models provide insight into how their respective pathways are integral to PCs.

KPT-330 is a chromosome region maintenance-1 (CRM1) inhibitor with antineoplastic activity. KPT-330 acts via the selective inhibition of nuclear export (SINE) approach—by modifying the essential CRM1-cargo binding residue C528, KPT-330 irreversibly inactivates CRM1-mediated nuclear export of cargo proteins, including growth regulation proteins. CRM1 co-immunoprecipitates with p27kip1, a protein whose constitutive expression causes cell cycle arrest in the G₁ phase that precedes differentiation. Upregulation of CRM1 and decreased levels of p27kip1 are observed in mucosal biopsies of patients with active Crohn's disease. Based on the results showing an increase in PC number and function with ENR+CD+KPT-330 treatment, CRM1 inhibition by KPT-330 may promote p27kip1-mediated cell cycle arrest to allow ISCs to transition first to a secretory cell progenitor, then to terminally differentiated PCs.

### Materials and Methods

### Crypt Isolation and Organoid Culture

Small intestinal crypts were isolated from C57BL/6 mice of both sexes, aged between three to six months in all experiments. Crypts were then cultured in a Matrigel culture system. Briefly, crypts were resuspended in basal culture medium (Advanced DMEM/F12 with 2 mM GlutaMAX and 10 mM HEPES; Thermo Fisher Scientific) at a 1:1 ratio with Corning™ Matrigel™ Membrane Matrix—GFR (Fisher Scientific) and plated at the center of each well of 24-well plates. Following Matrigel polymerization, 500 μL of small intestinal crypt culture medium (basal media plus 100× N2 supplement, 50×B27 supplement; Life Technologies, 500× N-acetyl-L-cysteine; Sigma-Aldrich) supplemented with growth factors EGF—E (50 ng/mL, Life Technologies), Noggin—N (100 ng/mL, PeproTech) and R-spondin 1—R (500 ng/mL, PeproTech) and small molecules CHIR99021—C (3 μM, LC Laboratories) and valproic acid—V (1 mM, Sigma-Aldrich) was added to each well. ROCK inhibitor Y-27632—Y (10 μM, R&D Systems) was added for the first 2 days of culture. Cells were cultured at 37° C. with 5% CO₂, and cell culture medium was changed every other day. After 6 days of culture, crypt organoids were isolated from Matrigel by mechanical dissociation. To expand enriched ISCs (ENR+CV/Y) or Paneth Cells (ENR+CD), organoids were cultured in 24-well plates, suspended in 40 uL 3-D gels (50-50 GFR MATRIGEL®, Basal culture media), with 500 uL of crypt media supplemented with necessary growth factors and small molecules. ROCK inhibitor (Y) was added for the first two days of ISC culture following reconstitution from cryopreservation or trypLE passaging to single cells. Cell culture medium was changed every other day. After 4 days of culture in ENR+CV, cell clusters were differentiated to PCs under the ENR+CD condition for 96-well short and long-term screens. For 384-well differentiation screens, 4-day ENR+CV clusters were passaged to single cells using trypLE, replated and expanded another 3 days in 3-D ENR+CVY and then passaged directly into screens.

Basal culture medium: Advanced DMEM/F12 with 2 mM GlutaMAX and 10 mM HEPES; Thermo Fisher Scientific.

### High-Throughput Screening: 96-Well Format

For 96-well plate high-throughput screening, 4-day differentiated (ENR+CD) cell clusters in 3D Matrigel were transferred to a “2.5-D” 96-well plate culture system. Briefly, cell culture gel and medium were homogenized via mechanical disruption and centrifuged at 300 g for 3 min at 4° C. Supernatant was removed and the pellet resuspended in basal culture medium repeatedly until the cloudy Matrigel was almost gone. On the last repeat, pellet was resuspended in basal culture medium, the number of cell clusters counted, and centrifuged at 300 g for 3 min at 4° C. The cell pellet was resuspended in ENR-CD medium and plated using a Tecan Evo liquid handler at the center of each well of 96-well plates prepared with a 45 uL polymerized 70% Matrigel (30% basal media) coating in each well. Plates were centrifuged at 50 g for 1 min at 4° C. to allow for cells to partially embed in Matrigel coating. At end time points (following 2 days in culture and 3 hours of stimulation), lysozyme secretion and cell viability were assessed using Lysozyme Assay Kit and CellTiter-Glo 3D Cell Viability Assay (Promega), respectively, according to the manufacturers' protocols. Briefly, 2.5D 96-well culture plates are spun at high speed (>2000 g) for 5 min at RT to pellet cell debris, then 25 μl of conditioned supernatant is removed from the top of each well and mixed with 75 μl lysozyme working solution using a black 96-well flat bottom plate (LYZ screen plate). The LYZ screen plate is covered, shaken for 10 min, incubated for 20 min at 37° C., then fluorescence measured (494 nm/518 nm). 25 μl CTG 3D is added to each well of the 2.5D culture plate, which is then shaken for 15 min before reading luminescence (integration time between 0.5 and 1 s). Replicate strictly standardized mean difference (SSMD) was used to determine the statistical effect size of each data point (treatment and dose grouped by replicate) relative to the untreated (basal non-stimulated) control using the formula for the robust uniformly minimal variance unbiased estimate (UMVUE) under the assumption that treatment has the same variance as the control:

$$SSMD = \frac{Γ ( \frac{n - 1}{2} )}{Γ ( \frac{n - 2}{2} )} \sqrt{\frac{2}{n - 1}} \frac{\overline{{d}_{l}}}{{s}_{i}} \qquad ( 1 )$$

where d̅ₗ and sᵢ are respectively the sample mean and standard deviation of dᵢⱼs where dᵢⱼ is the difference between the measured activity value (on the log scale) of the ith and the median value of a negative control in the jth plate, Γ(⋅) is a gamma function, and n is the replicate number. Formula adapted from analysis approaches for siRNA screens. Dose-response curves were fitted using GraphPad Prism 7.0d, using a least-squares dose-response fit to normalized assay values, with reported statistics derived from this fitting.

### High-Throughput Screening: 384-Well Plating and Assays

For 384-well plate high-throughput screening, ISC-enriched organoids were passaged and split to single cells with TyrpLE (Thermo Fisher Scientific) and cultured for 2-3 days in ENR+CVY prior to transfer to a “2.5-D” 384-well plate culture system. To prepare for “2.5D” plating, cell-laden Matrigel and media were homogenized via mechanical disruption and centrifuged at 300 g for 3 min at 4° C. Supernatant was removed and the pellet washed and spun in basal culture medium repeatedly until the cloudy Matrigel above the cell pellet was gone. On the final wash, pellet was resuspended in basal culture medium, the number of organoids counted, and the cell pellet was resuspended in ENR+CD medium at ˜7 clusters/μL. 384-well plates were first filled with 10 μL of 70% Matrigel (30% basal media) coating in each well using a Tecan Evo 150 Liquid Handling Deck, and allowed to gel at 37° C. for 5 minutes. Then 30 μL of cell-laden media was plated at the center of each well of 384-well plates with the liquid handler, and the plates were spun down at 50 g for 1 minute to embed organoids on the Matrigel surface. Compound libraries were pinned into prepped cell plates using 50 nL pins into 30 μL media/well. Cells were cultured at 37° C. with 5% CO₂ for six days in ENR+CD medium supplemented with the tested compounds with a media change at three days. On day six, lysozyme secretion and cell viability were assessed using Lysozyme Assay Kit (EnzChek) and CellTiter-Glo 3D Cell Viability Assay (Promega), respectively, according to the manufacturers' protocols. Briefly, screen plates were washed 3× with FluoroBrite basal media (2 mM GlutaMAX and 10 mM HEPES in FluoroBrite DMEM (Thermo Fisher Scientific)) using a BioTek 406 plate washer with 10 min incubations followed by a 1 min centrifugation at 200 g to settle media between washes. After removal of the third wash, 30 μL of non-stimulated FluoroBrite basal media was added to each screen well using a Tecan Evo 150 Liquid Handling Deck from a non-stimulated treatment master plate, and plates were incubated for 30 min at 37° C. After 30 minutes, the top 15 μL of media from each well of the screen plate was transferred to a non-stimulated LYZ assay plate containing 15 μL of 20× DQ LYZ assay working solution using a Tecan Evo 150 Liquid Handling Deck. The non-stimulated LYZ assay plate was covered, shaken for 10 min, incubated for 50 min at 37° C., then fluorescence measured (shake 10 s; 494 mm/518 nm) using a Tecan M1000 Plate Reader. After the media transfer to the non-stimulated LYZ assay plate, the remaining media was removed from the screen plate and 30 μL of Stimulated FluoroBrite basal media (supplemented with 10 μM CCh) was added to each screen well using a Tecan Evo 150 Liquid Handling Deck from a stimulated treatment master plate, and plates were incubated for 30 min at 37° C. After 30 minutes, the top 15 μL of media from each well of the screen plate was transferred to a stimulated LYZ assay plate containing 15 μL of 20× DQ LYZ assay working solution using a Tecan Evo 150 Liquid Handling Deck. The stimulated LYZ assay plate was covered, shaken for 10 min, incubated for 50 min at 37° C., then fluorescence measured (shake 10 s; 494 mm/518 nm) using a Tecan M1000 Plate Reader. Finally, 8 μL of CTG 3D was added to each well of the screen plate, which was shaken for 30 min at room temperature, then luminescence read (shake 10 s; integration time 0.5-1 s) to measure ATP.

### High-Throughput Screening: 384-Well Primary Screen Format and Analysis

Primary screens were performed using the Target Selective Inhibitor Library (Selleck Chemicals (Houston, Tex.) L3500) containing 433 compounds. Assays were performed in triplicate using four compound concentrations (0.08, 0.4, 2, and 10 μM). Each screen plate contained no cell controls, positive (ENR+CD+DMSO) controls, and positive controls with and without CCh stimulation for each LYZ assay.

A custom R script and pipeline was used for analysis of all screen results. Results (excel or .csv files) were converted into a data frame containing raw assay measurements corresponding to metadata for plate position, treatments, doses, cell type, and stimulation. Raw values were log₁₀ transformed, then a LOESS normalization was applied to each plate and assay to remove systematic error and column/row/edge effects using the formula:

\hat{x}ᵢⱼ=xᵢⱼ−(loess·fitᵢⱼ−median(loess·fitᵢⱼ)) (2)

where \hat{x}ᵢⱼ is the loess fit result, xᵢⱼ is the log₁₀ transformed at row i and column j, and loess. fitᵢⱼ is the value from loess smoothed data at row i and column j calculated using R loess function with span 1.

Following LOESS normalization, a plate-wise fold change (FC) calculation was performed on each well to normalize plates across the experiment. This was calculated by subtracting the median of the plate (as control) from the LOESS normalized values:

FCᵢⱼ=\hat{x}ᵢⱼ−median(\hat{x}ᵢⱼ) (3)

Replicate strictly standardized mean difference (SSMD) was used to determine the statistical effect size of each treatment in each assay (treatment and dose grouped by replicate, n=3) relative to the plate using the formula for the robust uniformly minimal variance unbiased estimate (UMVUE):

$$SSMD = \frac{Γ ( \frac{n - 1}{2} )}{Γ ( \frac{n - 2}{2} )} \sqrt{\frac{2}{n - 1}} \frac{{\overline{d}}_{i}}{\sqrt{{w}_{i} {s}_{i}^{2} + {w}_{0} {s}_{0}^{2}}} \qquad ( 4 )$$

where dᵢ and sᵢ are respectively the sample mean and standard deviation of dᵢⱼs where dᵢⱼ is the FC for the ith treatment on the jth plate. Γ(⋅) is a gamma function. s₀² is an adjustment factor equal to the median of all sᵢ²s to provide a more stable estimate of variance. wᵢ and w₀ are weights equal to 0.5 with the constraint of wᵢ+w₀=1. n is the replicate number.

Mean FC (the arithmetic mean of all samples grouped by treatment and dose across replicates) was used to determine the z-score for each treatment and dose with the formula:

$$z = \frac{meanFC}{{SD}_{pop}} \qquad ( 5 )$$

where SDₚₒₚ is the standard deviation of all mean-FC's.

All calculated statistics were combined in one finalized data table and exported as a .csv file for hit identification. A primary screen “hit” was defined as having SSMDs for both LYZ assays greater than the optimal critical value (β_{α}_{₁}=0.997) and being in the top 10% of a normal distribution of FC values for both assays with a z-score cutoff>1.282. β_{α}_{₁} was determined by minimizing the false positive (FPL) and false negative (FNL) levels for up-regulation SSMD-based decisions by solving for the intersection of the formulas:

$$\begin{matrix}{F}_{t ( n - 1 , \sqrt{n} {β}_{2} )} ( \frac{{βα}_{1}}{k} ) = 1 - FPL & ( 6 ) \\ and FNL = {F}_{t ( n - 1 , \sqrt{n} {β}_{1} )} ( \frac{{βα}_{1}}{k} ) & ( 7 ) \\ where k = \sqrt{\frac{1}{n}} & ( 8 )\end{matrix}$$

where F_{t(n-1,\sqrt{n}β)}(⋅) is the cumulative distribution function of non-central t-distribution t(n−1, \sqrt{n}β) and n is the number of replicates, β₂ is a SSMD bound for FPL of 0.25 (at least very weak effect), and β₁ is a SSMD bound for FNL of 3 (at least strong effect).

Hit treatments were thus selected to have a well-powered statistical effect size as well as a strong biological effect size. Optimal dose per hit treatment was determined by SSMD for both LYZ assays.

High-Throughput Screening: 384-Well Secondary Screen Format and Early Vs. Full Treatment Analysis

Confirmatory secondary screening with primary hits was performed using the above 384-well plate method, in parallel with time point investigation (early vs. full). The screen was conducted with 4-plate replicates for both ENR+CD and ENR conditions. Media was supplemented with compound early (day 1-3 only n=4 well replicates per dose) or full (day 1-6 n=8 well replicates per dose) at four different doses: two-fold above, two-fold below, and four-fold below the optimal final dose for each respective treatment. Additionally, each plate carried a large number of ENR+DMSO or ENR+CD+DMSO (vehicle) control wells (n=100 for ATP, and n=25 for LYZ.NS and LYZ.S) for robust normalization. ATP, non-stimulated lysozyme activity and CCh-stimulated lysozyme activity was again measured and the collected data was again processed in a custom R-script, per primary screen with slight modification. Values were log₁₀ transformed, and a plate-wise FC was calculated for each well based on the median value of ENR+CD+DMSO (vehicle) control wells to normalize plate to plate variability. The following formula was used:

FCᵢⱼ=xᵢⱼ−median(xₚₒₛ) (9)

Where xᵢⱼ is the log₁₀ transformed value at row i and column j, and xₚₒₛ are the values of the positive control wells. For the ATP assay, all vehicle-only wells were used as the control. For the LYZ.NS assay, non-stimulated vehicle only wells were used. For the LYZ.S assay, vehicle only wells that were non-stimulated in the LYZ.NS assay then stimulated in the LYZ.S were used.

Once normalized, the replicate SSMD was calculated using formula (4) to quantify statistical effect size with 8 replicate differences taken relative to the respective plate ENR+DMSO or ENR+CD+DMSO median value. A primary hit was considered validated when SSMDs for both LYZ assays was greater than the optimal critical value (β_{α}_{₁}) of 0.889. β_{α}_{₁} was determined using formula (6) with an FPL error of 0.05 for a more stringent cut off, FNL was not considered. Optimal doses were chosen for treatments with multiple validated doses by taking the most potent (highest mean fold change relative to ENR+CD control) dose in both LYZ assays.

### Flow Cytometry Profiling of Organoids in “2.5-D”

For flow cytometry profiling of the 6 validate small molecules, ISC-enriched ‘small clusters’ in 3D Matrigel culture were passaged to a “2.5D” 96-well plate culture system for six days of ENR, or ENR+CD+drug culture in the same manner as described previously with the exception of plating in 96-well plates prepared with a polymerized 70% Matrigel coating in each well. Plates were centrifuged at 50 g for 1 min at 4° C. to allow for cells to partially embed in Matrigel coating. Drugs were pinned into their respective wells using the Tecan from a drug stamp plate. Media was changed at day three, including pinning of the drug treatments. At day six, cells were washed 3× with basal media, then harvested from Matrigel by mechanical disruption in TrypLE Express to remove Matrigel and dissociate organoids to single cells. After vigorous pipetting and incubation at 37° C. for 20 mins, dissociated organoids were strained through a 96-well filter plate with a 30-40 μm filter (Pall) into an ultra low-bind 96-well plate (Costar) by centrifuging at 300×g for 3 mins at 4° C. The cell filtrate was centrifuged again at 300×g for 3 mins at 4° C. to pellet the cells. Cell pellets were resuspended in FACS buffer (2% FBS in PBS0), then transferred to an ultra low-bind 96-well plate for flow prep. Cells were stained with Zombie-violet viability dye (BioLegend) at 100× for viability staining and/or antibody staining solution. FITC-conjugated antibody for lysozyme and APC-conjugated antibody for CD24 were used at 100× dilution (BioLegend). Flow cytometry was performed using a LSR Fortessa (BD; Koch Institute Flow Cytometry Core at MIT). Flow cytometry data was analyzed using FlowJo X v10.1 software.

### REFERENCES

1. Turnbaugh, P. J. et al. An obesity-associated gut microbiome with increased capacity for energy harvest. Nature 444, 1027-31 (2006).
2. Everard, A. et al. Cross-talk between Akkermansia muciniphila and intestinal epithelium controls diet-induced obesity. Proc. Natl. Acad. Sci. 110, 9066-9071 (2013).
3. Sato, T. et al. Long-term expansion of epithelial organoids from human colon, adenoma, adenocarcinoma, and Barrett's epithelium. Gastroenterology 141, 1762-72 (2011).
4. Bhatia, S. N. & Ingber, D. E. Microfluidic organs-on-chips. Nat. Biotechnol. 32, 760-772 (2014).
5. Clevers, H. Modeling Development and Disease with Organoids. Cell 165, 1586-1597 (2016).
6. Drost, J. et al. Use of CRISPR-modified human stem cell organoids to study the origin of mutational signatures in cancer. Science (80-.). 238, eaao3130 (2017).
7. Molodecky, N. a et al. Increasing incidence and prevalence of the inflammatory bowel diseases with time, based on systematic review. Gastroenterology 142, 46-54.e42; quiz e30 (2012).
8. Kaser, A. & Blumberg, R. S. ATG16L1 Crohn's disease risk stresses the endoplasmic reticulum of Paneth cells. Gut 2013-2015 (2013). doi:10.1136/gutjn1-2013-306103
9. Adolph, T. E. et al. Paneth cells as a site of origin for intestinal inflammation. Nature 503, 272-6 (2013).
10. Kaser, A. et al. XBP1 links ER stress to intestinal inflammation and confers genetic risk for human inflammatory bowel disease. Cell 134, 743-56 (2008).
11. Hodin, C. M. et al. Reduced Paneth cell antimicrobial protein levels correlate with activation of the unfolded protein response in the gut of obese individuals. J. Pathol. 225, 276-284 (2011).
12. Ayabe, T. et al. Secretion of microbicidal α-defensins by intestinal Paneth cells in response to bacteria. Nat. Immunol. 1, 113-118 (2000).
13. Farin, H. F. et al. Paneth cell extrusion and release of antimicrobial products is directly controlled by immune cell-derived IFN-γ. J. Exp. Med. 211, 1393-405 (2014).
14. VanDussen, K. L. et al. Notch signaling modulates proliferation and differentiation of intestinal crypt base columnar stem cells. Development 139, 488-497 (2012).
15. Bevins, C. L. & Salzman, N. H. Paneth cells, antimicrobial peptides and maintenance of intestinal homeostasis. Nat. Rev. Microbiol. 9, 356-68 (2011).
16. Peterson, L. W. & Artis, D. Intestinal epithelial cells: regulators of barrier function and immune homeostasis. Nat. Rev. Immunol. 14, 141-153 (2014).
17. Xavier, R. J. & Podolsky, D. K. Unravelling the pathogenesis of inflammatory bowel disease. Nature 448, 427-34 (2007).
18. McGuckin, M. a, Eri, R., Simms, L. a, Florin, T. H. J. & Radford-Smith, G. Intestinal barrier dysfunction in inflammatory bowel diseases. Inflamm. Bowel Dis. 15, 100-13 (2009).
19. Khor, B., Gardet, A. & Xavier, R. J. Genetics and pathogenesis of inflammatory bowel disease. Nature 474, 307-17 (2011).
20. Liu, T.-C. et al. Paneth cell defects in Crohn's disease patients promote dysbiosis. JCI insight 1, e86907 (2016).
21. Gassler, N. Paneth cells in intestinal physiology and pathophysiology. World J. Gastrointest. Pathophysiol. 8, 150-160 (2017).
22. Rivollier, A., He, J., Kole, A., Valatas, V. & Kelsall, B. L. Inflammation switches the differentiation program of Ly6Chi monocytes from antiinflammatory macrophages to inflammatory dendritic cells in the colon. J. Exp. Med. 209, 139-55 (2012).
23. Saenz, S. A. et al. IL25 elicits a multipotent progenitor cell population that promotes T(H)2 cytokine responses. Nature 464, 1362-6 (2010).
24. Siracusa, M. C. et al. TSLP promotes interleukin-3-independent basophil haematopoiesis and type 2 inflammation. Nature 477, 229-33 (2011).
25. Sonnenberg, G. F., Monticelli, L. A., Elloso, M. M., Fouser, L. A. & Artis, D. CD4(+) lymphoid tissue-inducer cells promote innate immunity in the gut. Immunity 34, 122-34 (2011).
26. Velcich, A. et al. Colorectal cancer in mice genetically deficient in the mucin Muc2. Science 295, 1726-9 (2002).
27. Sherman, M. P., Bennett, S. H., Hwang, F. F. Y., Sherman, J. & Bevins, C. L. Paneth cells and antibacterial host defense in neonatal small intestine. Infect. Immun. 73, 6143-6 (2005).
28. McElroy, S. J., Underwood, M. A. & Sherman, M. P. Paneth cells and necrotizing enterocolitis: a novel hypothesis for disease pathogenesis. Neonatology 103, 10-20 (2013).
29. White, J. R., Gong, H., Pope, B., Schlievert, P. & McElroy, S. J. Paneth-cell-disruption-induced necrotizing enterocolitis in mice requires live bacteria and occurs independently of TLR4 signaling. Dis. Model. Mech. 10, 727-736 (2017).
30. Tanner, S. M. et al. Pathogenesis of necrotizing enterocolitis: modeling the innate immune response. Am. J. Pathol. 185, 4-16 (2015).
31. Eriguchi, Y. et al. Graft-versus-host disease disrupts intestinal microbial ecology by inhibiting Paneth cell production of α-defensins. Blood 120, 223-31 (2012).
32. Vijay-Kumar, M. et al. Metabolic Syndrome and Altered Gut Microbiota in Mice Lacking Toll-Like Receptor 5. Science (80-.). 328, 228-231 (2010).
33. Hayase, E. et al. R-Spondin1 expands Paneth cells and prevents dysbiosis induced by graft-versus-host disease. J. Exp. Med. 214, 3507-3518 (2017).
34. Han, T. et al. R-Spondin chromosome rearrangements drive Wnt-dependent tumour initiation and maintenance in the intestine. Nat. Commun. 8, 15945 (2017).
35. Sansom, O. J. et al. Loss of Apc in vivo immediately perturbs Wnt signaling, differentiation, and migration. Genes Dev. 18, 1385-90 (2004).
36. Okubo, T. & Hogan, B. L. M. Hyperactive Wnt signaling changes the developmental potential of embryonic lung endoderm. J. Biol. 3, 11 (2004).
37. Zhou, X. et al. R-Spondin1/LGR5 Activates TGFβ Signaling and Suppresses Colon Cancer Metastasis. Cancer Res. 77, 6589-6602 (2017).
38. Kim, K.-A. et al. Mitogenic influence of human R-spondin1 on the intestinal epithelium. Science 309, 1256-9 (2005).
39. Fre, S. et al. Notch signals control the fate of immature progenitor cells in the intestine. Nature 435, 964-8 (2005).
40. Jensen, J. et al. Control of endodermal endocrine development by Hes-1. Nat. Genet. 24, 36-44 (2000).
41. VanDussen, K. L. & Samuelson, L. C. Mouse atonal homolog 1 directs intestinal progenitors to secretory cell rather than absorptive cell fate. Dev. Biol. 346, 215-23 (2010).
42. Horvath, P. et al. Screening out irrelevant cell-based models of disease. Nat. Rev. Drug Discov. 15, 751-769 (2016).
43. Kola, I. & Landis, J. Can the pharmaceutical industry reduce attrition rates? Nat. Rev. Drug Discov. 3, 711-716 (2004).
44. Hay, M., Thomas, D. W., Craighead, J. L., Economides, C. & Rosenthal, J. Clinical development success rates for investigational drugs. Nat. Biotechnol. 32, 40-51 (2014).
45. Dionne, S., Laberge, S., Deslandres, C. & Seidman, E. G. Modulation of cytokine release from colonic explants by bacterial antigens in inflammatory bowel disease. Clin. Exp. Immunol. 133, 108-114 (2003).
46. Neurath, M. F. Cytokines in inflammatory bowel disease. Nat. Rev. Immunol. 14, 329-342 (2014).
47. Rumio, C. et al. Induction of Paneth cell degranulation by orally administered Toll-like receptor ligands. J. Cell. Physiol. 227, 1107-1113 (2012).
48. Mastroianni, J., Lu, W., Selsted, M. & Ouellette, A. Differential Susceptibility of Bacteria to Mouse Paneth Cell a-Defensins under Anaerobic Conditions. Antibiotics 3, 493-508 (2014).
49. Satoh, Y., Ishikawa, K., Oomori, Y., Takeda, S. & Ono, K. Bethanechol and a G-protein activator, NaF/AlCl3, induce secretory response in Paneth cells of mouse intestine. Cell Tissue Res. 269, 213-220 (1992).
50. Zhang, X. D. in Optimal High-Throughput Screening 154-188 (Cambridge University Press, 2011). doi:10.1017/CB09780511973888.009
51. Fischer, J. M. et al. Single cell lineage tracing reveals a role for TgfβR2 in intestinal stem cell dynamics and differentiation. Proc. Natl. Acad. Sci. 201611980 (2016). doi:10.1073/pnas.1611980113
52. Rhee, C. K. et al. Effect of nilotinib on bleomycin-induced acute lung injury and pulmonary fibrosis in mice. Respiration. 82, 273-87 (2011).
53. Dervis Hakim, G. et al. Mucosal healing effect of nilotinib in indomethacin-induced enterocolitis: A rat model. World J. Gastroenterol. 21, 12576-85 (2015).
54. Ataca, P. et al. Nilotinib-mediated mucosal healing in a rat model of colitis. World J. Gastroenterol. 19, 6237-44 (2013).
55. Li, J. [Molecular mechanism of gastrointestinal stromal tumors and progress in drug research]. Zhonghua Wei Chang Wai Ke Za Zhi 19, 1316-1320 (2016).
56. Christensen, J. G. et al. A selective small molecule inhibitor of c-Met kinase inhibits c-Met-dependent phenotypes in vitro and exhibits cytoreductive antitumor activity in vivo. Cancer Res. 63, 7345-55 (2003).
57. Joosten, S. P. J. et al. MET Signaling Mediates Intestinal Crypt-Villus Development, Regeneration, and Adenoma Formation and Is Promoted by Stem Cell CD44 Isoforms. Gastroenterology 153, 1040-1053.e4 (2017).
58. Kumar, N., Goldminz, A. M., Kim, N. & Gottlieb, A. B. Phosphodiesterase 4-targeted treatments for autoimmune diseases. BMC Med. 11, 96 (2013).
59. Christensen, S. B. et al. 1,4-Cyclohexanecarboxylates: potent and selective inhibitors of phosophodiesterase 4 for the treatment of asthma. J. Med. Chem. 41, 821-35 (1998).
60. Videla, S. Selective Inhibition of Phosphodiesterase-4 Ameliorates Chronic Colitis and Prevents Intestinal Fibrosis. J. Pharmacol. Exp. Ther. 316, 940-945 (2005).
61. Kamei, K. et al. Prophylactic effect of irsogladine maleate against indomethacin-induced small intestinal lesions in rats. Dig. Dis. Sci. 53, 2657-66 (2008).
62. Schewe, M. et al. Secreted Phospholipases A2 Are Intestinal Stem Cell Niche Factors with Distinct Roles in Homeostasis, Inflammation, and Cancer. Cell Stem Cell 19, 38-51 (2016).
63. Yan, L. et al. Chromosome region maintenance-1 (CRM1) regulates apoptosis of intestinal epithelial cells via p27kip1 in Crohn's disease. Clin. Res. Hepatol. Gastroenterol. 41, 445-458 (2017).
64. Lloyd, R. V et al. p27kip1: a multifunctional cyclin-dependent kinase inhibitor with prognostic significance in human cancers. Am. J. Pathol. 154, 313-23 (1999).
65. Zhang, X. D. Illustration of SSMD, z score, SSMD*, z* score, and t statistic for hit selection in RNAi high-throughput screens. J. Biomol. Screen. 16, 775-85 (2011).
66. Mpindi, J. P. et al. Impact of normalization methods on high-throughput screening data with high hit rates and drug testing with dose-response data. Bioinformatics 31, 3815-3821 (2015).
67. Zhang, X. D. in Optimal High-Throughput Screening 83-108 (Cambridge University Press, 2011). doi:10.1017/CB09780511973888.007

### Example 2: Validation of XPO1 Inhibition as a Means to Enhance Paneth Cell Differentiation

Of the six promising lead small molecules identified in Example 1, KPT-330 appears to most significantly enhance Paneth cell differentiation, and as such we sought to better understand the mechanism through which KPT-330 may be acting, whether by canonical XPO1 inhibition, or other means.

### Results

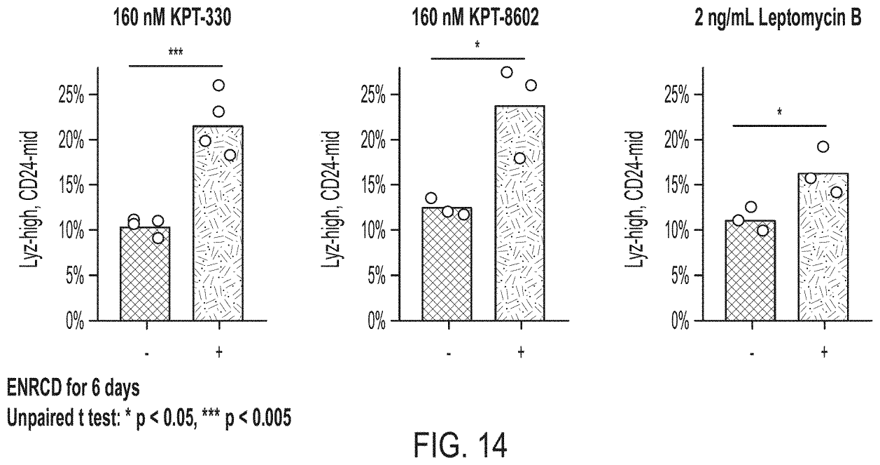

To address whether enhanced Paneth cell differentiation within ENR+CD organoids following KPT-330 treatment is stemming from the known mechanism of XPO1 inhibition, or a potential off-target or non-canonical effect, we repeated organoid differentiation with two additional known XPO1 inhibitors, KPT-8602 and Leptomycin B. As measured by flow cytometry (per the same gating strategy employed in Example 1), consistent, statistically significant increases in Paneth cell representation were observed following treatment with KPT-330, KPT-8602, and Leptomycin B (FIG. 14, wherein “+” denotes administration of the small molecule and “−” denotes the absence of that compound in the media).

### Discussion

KPT-330 administration drives Paneth cell differentiation through canonical XPO1 inhibition, as confirmed by parallel assessment with additional known XPO1 inhibitors KPT-8602 and Leptomycin B, which lead to similar, statistically significant increases in the Paneth cell fraction of within ENR+CD differentiated organoids.

### Materials and Methods

### Antibodies and Reagents

An antibody against lysozyme was purchased from abcam (Cambridge, Mass., ab108508). KPT-330 (S7252) and KPT-8602 (S8397) were purchased from Selleck Chemicals (Houston, Tex.), and leptomycin B was purchased from Cayman Chemical (Ann Arbor, Mich.; 10004976).

### Flow Cytometry Analysis—3D Culture

ISC-enriched organoids cultured in 3D Matrigel with ENRCV media were passaged to 24-well plate in 3D Matrigel with ENRCV. At day zero, media were replaced to ENRCD with or without indicated compounds, and media were replaced every other day. At day six, cells were washed with basal media, and then harvested from Matrigel by mechanical disruption in TrypLE Express (ThermoFisher, 12605010) to remove Matrigel and dissociate organoids to single cells. After vigorous pipetting and incubation at 37° C. for 20 min, dissociated organoids were strained through a 35 μm cell strainer into a tube (Falcon, 352235). The cell filtrate was centrifuged again at 300×g for 3 min at 4° C. to pellet the cells. Cell pellets were resuspended in FACS buffer (2% FBS in PBS), and then transferred to an ultra low-bind 96-well plate (Corning, 7007). Cells were stained with Zombie-violet viability dye (BioLegend, 423107) at 100× for viability staining and/or antibody staining solution. FITC-conjugated antibody for lysozyme (Dako, F0372) and APC-conjugated antibody for CD24 (BioLegend, 138505) were used at 100× dilution. Flow cytometry was performed using an LSR Fortessa (BD; Koch Institute Flow Cytometry Core at MIT). The data were analyzed using FlowJo v10 software.

### Example 3: XPO1 Inhibition is not Dependent on Wnt or Notch Pathway Modulation to Induce Paneth Cell Differentiation

In continuing our investigation of the biological mechanism of small molecule inhibitors of XPO1 in vitro on the differentiation of ISCs into Paneth cells, the interdependence of XPO1 inhibition with Wnt and Notch pathway modulation in driving enhanced secretory differentiation was assessed.

### Results

Using the high-fidelity PC model (see Example 1) in the traditional 3D enteroid culture system, we differentiated ISC-enriched organoids from one biological donor and then sought to assess to what extent XPO1 inhibition is interdependent on Wnt agonism and Notch antagonism to drive secretory (Paneth) cell differentiation. We assessed this through studies of bulk transcripts, protein, and functional assays.

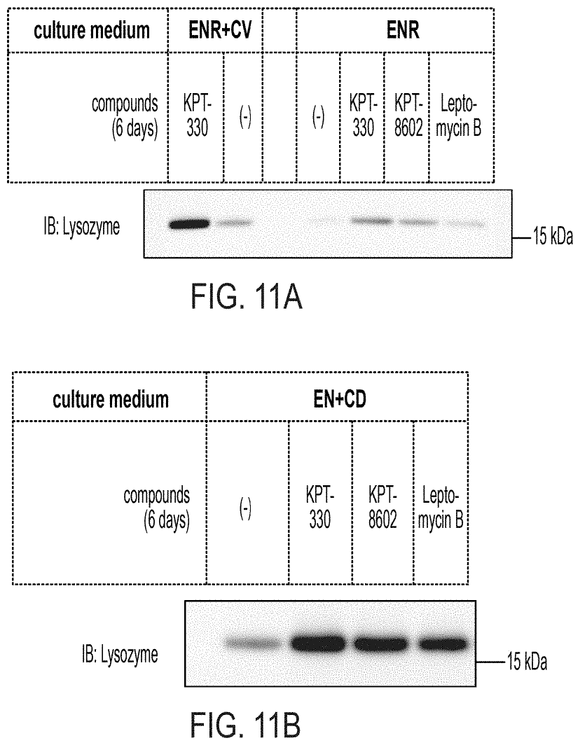

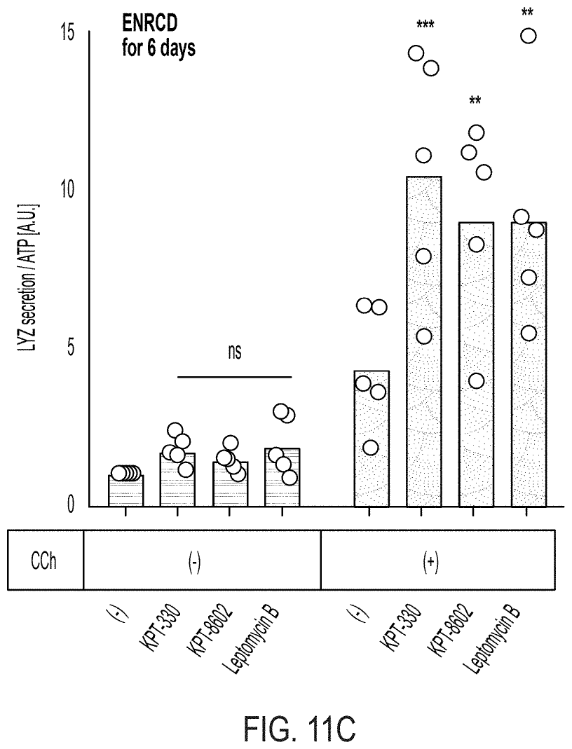

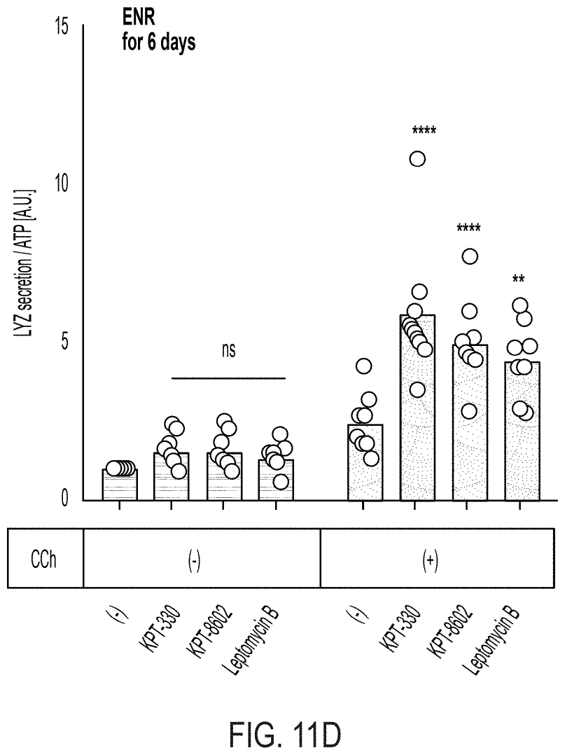

Assaying bulk protein extracted from organoids differentiated under either ENR+CV, ENR+CD, or ENR for LYZ, it's apparent that the addition of XPO1 inhibitors KPT-330, KPT-8602, and Leptomycin B all appreciably increase LYZ abundance on a per-mass protein normalized basis (FIGS. 11A-B). Additionally, for both ENR and ENR+CD following 6-day differentiation, functional lysozyme secretion is greatly enhanced when XPO1 inhibitors are added (FIGS. 11C-D).

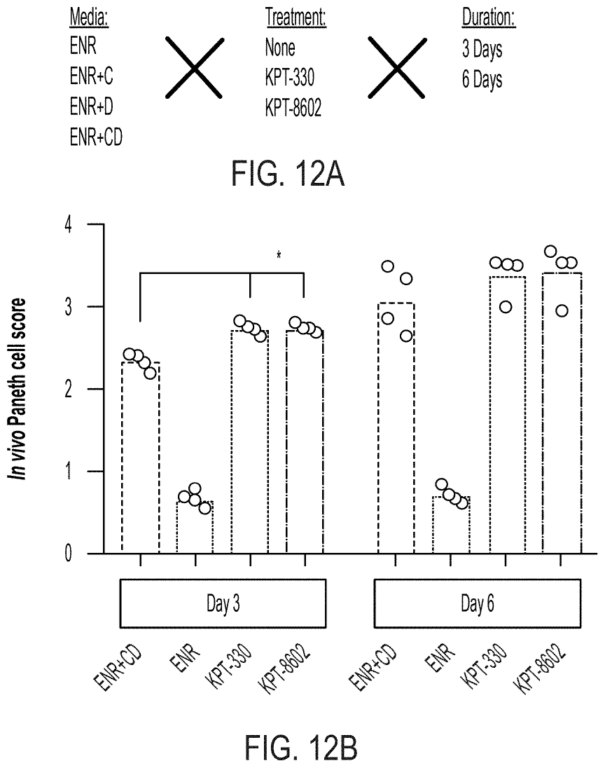

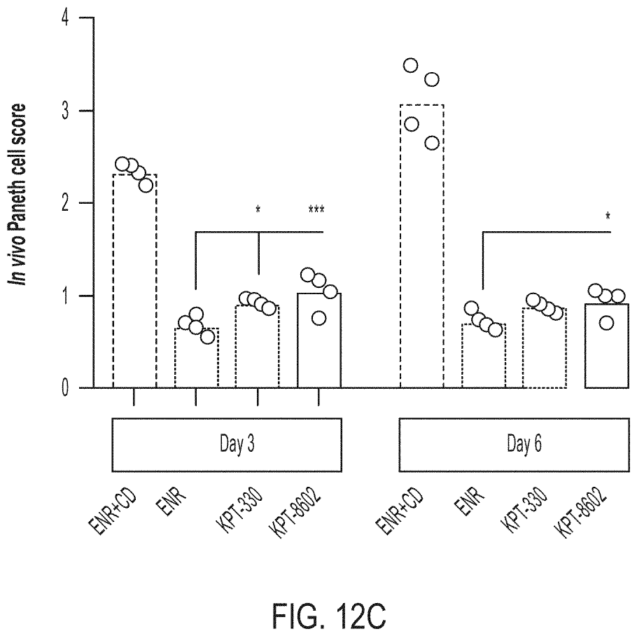

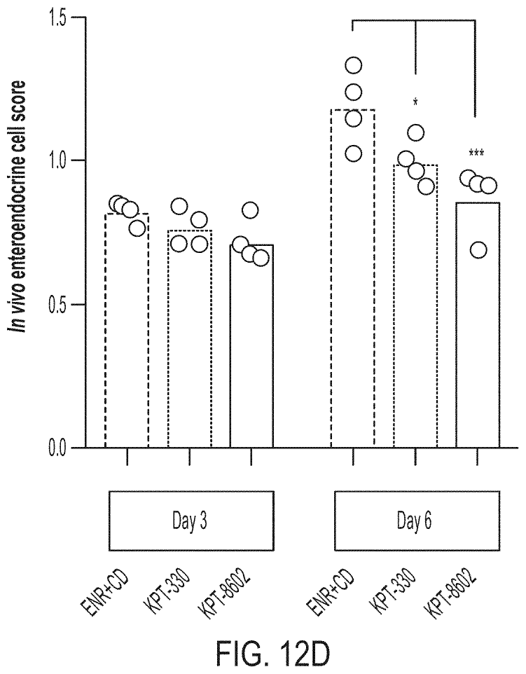

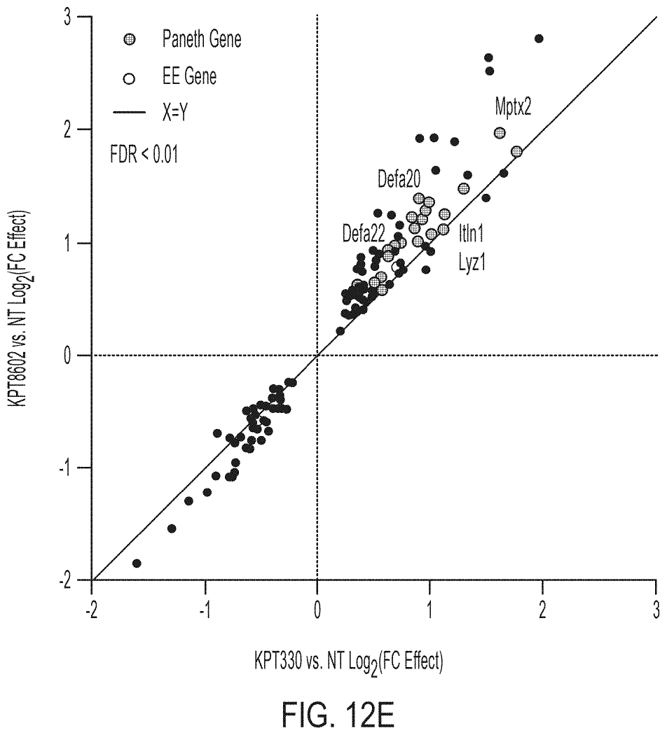

To further assess the potential interaction of Wnt agonist CHIR99021 (C) and Notch inhibitor DAPT (D) with XPO1 inhibitors, we carried out an experiment where we assessed bulk organoid population transcriptomes (with RNA-seq) from multiple variations on growth media, XPO1 inhibitor, and duration of treatment (FIG. 12A). To characterize the resulting cell phenotypes, we used aggregate scores of geneset enrichment (based on module scoring as implemented in⁷) from the in vivo Paneth and related enteroendocrine cell populations (genesets taken from⁶). We see that again for both ENR+CD treated (FIG. 12B) and ENR treated (FIG. 12C) organoid populations, XPO1 inhibition drives enrichment in Paneth cell defining genes, principally at 3 days of treatment. Interestingly, we see that in ENR+CD organoids, the enteroendocrine score greatly decreases following XPO1 inhibition for 6 days, suggesting that XPO1 inhibition may drive a lineage choice between enteroendocrine and Paneth cells during ISC differentiation (FIG. 12D). Finally, to more globally determine the key transcriptomic changes driven by XPO1 inhibition, we used the DESeq2 R package to model differential gene expression following 3 days of treatment when all media conditions are controlled. The majority of significantly up-regulated genes for both KPT-330 and KPT-8602 relative to no treatment are known Paneth cell lineage-defining genes, demonstrating that XPO1 inhibition can independently drive ISC differentiation into the Paneth cell lineage in an organoid model (FIG. 12E).

In total, this suggests that the small molecule XPO1 inhibitors tested in this organoid-derived PC-differentiation experiment are able to drive PC differentiation and provide functional improvements (lysozyme secretion) independently of Wnt agonism and Notch antagonism.

### Discussion

### The Tested Set of XPO-1 Inhibiting Small Molecules (KPT-330, KPT-8602, Leptomycin B) Robustly Increases Paneth Cell Differentiation in Organoids Independently of WNT and Notch Signaling

Selective inhibition of nuclear export (SINE) approach—by modifying the essential XPO1-cargo binding residue C528, KPT-330 and KPT-8602 irreversibly inactivates XPO1-mediated nuclear export of cargo proteins, including growth regulation proteins. As well, XPO1 co-immunoprecipitates with p27kip1, a protein whose constitutive expression causes cell cycle arrest in the G₁ phase that precedes differentiaton^{8,9}. Upregulation of XPO1 and decreased levels of p27kip1 are observed in mucosal biopsies of patients with active Crohn's disease⁸. Given these findings and the data presented herein, we propose that XPO-1 inhibition is a potent and specific enhancer of Paneth cell differentiation from intestinal stem cells, and act independently of the Wnt and Notch signaling pathways, while also synergizing with those pathways to enhance epithelial secretory cell differentiation.

### Materials and Methods

### Organoid Culture

See Example 1, Materials and Methods, Crypt isolation and organoid culture.

### Western Blotting

ISC-enriched organoids cultured in 3D Matrigel with ENRCV media were passaged to 24-well plate in 3D Matrigel with ENRCV. At day zero, media were replaced to ENR or ENRCD with or without indicated compounds, and media were replaced every other day. At day six, cells were harvested from Matrigel by mechanical disruption and suspended in basal media. Cell pellets were lysed with Pierce® IP lysis buffer (ThermoFisher, 87787) containing Halt™ Protease Inhibitor Cocktail, EDTA-Free (ThermoFisher, 87785) after 3-minute centrifuge at 300×g at 4° C. Cell extracts were resolved by NuPAGE® SDS-PAGE Gel system (ThermoFisher) and electroblotted onto polyvinylidene difluoride membranes using Criterion™ Blotter (Biorad). The membranes were blocked with 2% Blotting-Grade Blocker (Biorad, 1706404) in TBS-T (50 mM Tris-HCl, 150 mM NaCl, and 0.1% Tween 20, pH 8.0) and then probed with appropriate antibodies. Detection was performed using ECL Select™ Western Blotting Detection Reagent (Amersham, 45-000-999) and ImageQuant LAS4000 (GE Healthcare).

### Lysozyme Assay—3D Culture

ISC-enriched organoids cultured in 3D Matrigel with ENRCV media were passaged to 48-well plate in 3D Matrigel with ENRCV. At day zero, media were replaced to ENR or ENRCD with or without indicated compounds, and media were replaced every other day. At day six, cells were washed twice with basal media and treated with carbamylcholine chloride (Sigma, C4382) for 3 hours. Media were collected and lysozyme activity was measured by EnzChek Lysozyme Assay Kit (ThermoFisher, E22013). Simultaneously, cell viability was measured by CellTiter-Glo® 3D Cell Viability Assay (Promega, G9681).

### Population RNA-Sequencing

Population RNA-seq was performed using a derivative of the Smart-Seq2 protocol for single cells^{1,2}. In brief, organoid media was aspirated and RLT+BME (Qiagen) was added to each well, and plate shaken for 30 minutes to fully lyse. Lysate was aliquoted into 4 identical fractions and stored at −80° C. until reverse transcription. RNA lysate was thawed and cleaned with a 2.2×SPRI ratio using Agencourt RNAClean XP beads (Beckman Coulter, A63987). RNA-seq was performed on a bulk population of sorted basal cells using Smart-Seq2 chemistry, starting with a 2.2×SPRI ratio cleanup. After oligo-dT priming, Maxima H Minus Reverse Transcriptase (ThermoFisher EP0753) was used to synthesize cDNA with an elongation step at 52° C. before PCR amplification (15 cycles for tissue, 18 cycles for sorted basal cells) using KAPA HiFi PCR Mastermix (Kapa Biosystems KK2602). Sequencing libraries were prepared using the Nextera XT DNA tagmentation kit (Illumina FC-131-1096) with 250 pg input for each sample. Libraries were pooled post-Nextera and cleaned using Agencourt AMPure SPRI beads with successive 0.7× and 0.8× ratio SPRIs and sequenced with an Illumina 75 Cycle NextSeq500/550v2 kit (Illumina FC-404-2005) with loading density at 2.2 pM, with paired end 35 cycle read structure. Samples were sequenced at an average read depth of 8.44 million reads per sample and a total of 96 samples.

Organoid samples were aligned to the Mm10 genome and transcriptome using STAR³ and RSEM⁴. Differential expression analysis was conducted using DESeq2 package for R⁵. Genes regarded as significantly differentially expressed were determined based on an adjusted P value using the Benjamini-Hochberg procedure to correct for multiple comparisons with a false discovery rate <0.01. For module scoring with the in vivo Paneth and enteroendocrine cell-defining genes, we used previously published⁶ gene sets and computed per-sample module scores through the Seurat package⁷.

### REFERENCES

1. Trombetta, J. J. et al. Preparation of single-cell RNA-Seq libraries for next generation sequencing. Curr. Protoc. Mol. Biol. 2014, 4.22.1-4.22.17 (2014).
2. Ordovas-Montanes, J. et al. Allergic inflammatory memory in human respiratory epithelial progenitor cells. Nature 560, 649-654 (2018).
3. Dobin, A. et al. STAR: Ultrafast universal RNA-seq aligner. Bioinformatics 29, 15-21 (2013).
4. Li, B. & Dewey, C. N. RSEM: accurate transcript quantification from RNA-Seq data with or without a reference genome. BMC Bioinformatics 12, 323 (2011).
5. Love, M. I., Huber, W. & Anders, S. Moderated estimation of fold change and dispersion for RNA-seq data with DESeq2. Genome Biol. 15, 1-21 (2014).
6. Mead, B. E. et al. Harnessing single-cell genomics to improve the physiological fidelity of organoid-derived cell types. BMC Biol. 16, 62 (2018).
7. Satija, R., Farrell, J. A., Gennert, D., Schier, A. F. & Regev, A. Spatial reconstruction of single-cell gene expression data. Nat. Biotechnol. 33, 495-502 (2015).
8. Yan, L. et al. Chromosome region maintenance-1 (CRM1) regulates apoptosis of intestinal epithelial cells via p27kip1 in Crohn's disease. Clin. Res. Hepatol. Gastroenterol. 41, 445-458 (2017).
9. Lloyd, R. V et al. p27kip1: a multifunctional cyclin-dependent kinase inhibitor with prognostic significance in human cancers. Am. J. Pathol. 154, 313-23 (1999).

### INCORPORATION BY REFERENCE; EQUIVALENTS

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A method of differentiating leucine-rich repeat-containing G-protein coupled receptor 5-positive (LGR5⁺) intestinal cells, the method comprising contacting LGR5⁺ cells with an inhibitor of exportin 1 (XPO1), thereby producing functionally differentiated intestinal cells.

2. The method of claim 1, wherein the inhibitor of XPO1 is KPT-330.

3. The method of claim 1, wherein the inhibitor of XPO1 is KPT-8602.

4. The method of claim 1, wherein the inhibitor of XPO1 is Leptomycin B.

5. The method of claim 1, further comprising contacting the LGR5⁺ cells with a Wnt agonist.

6. The method of claim 5, wherein the Wnt agonist is CHIR99021.

7. The method of claim 1, further comprising contacting the LGR5+ cells with a Notch inhibitor.

8. The method of claim 7, wherein the Notch inhibitor is DAPT.

9. The method of claim 1, wherein the functionally differentiated intestinal cells are Paneth cells.

10. The method of claim 1, wherein the functionally differentiated intestinal cells are CD24-mid/LYZ⁺ cells.

11. The method of claim 10, wherein the CD24-mid/LYZ⁺ cells are Paneth cells.

12. The method of claim 1, wherein the LGR5⁺ intestinal cells are intestinal stem cells.

13. The method of claim 1, wherein the functionally differentiated intestinal cells secrete greater quantities of lysozyme compared to the LGR5⁺ intestinal cells.

14. The method of claim 1, wherein the functionally differentiated intestinal cells express any combination of one or more of human lysozyme (LYZ), a human alpha defensin (DEFA), human matrix metalloproteinase-7 (MMP-7), and cluster of differentiation 24 (CD24).

15. The method of claim 1, wherein the functionally differentiated intestinal cells express human lysozyme (LYZ).

16. The method of claim 1, wherein the functionally differentiated intestinal cells express a human alpha defensin (DEFA).

17. The method of claim 16, wherein the human alpha defensin is human alpha defensin 5 (DEFA5) or human alpha defensin 6 (DEFA6).

18. The method of claim 1, wherein the functionally differentiated intestinal cells express mRNA for any combination of one or more of human lysozyme (LYZ), a human alpha defensin (DEFA), human matrix metalloproteinase-7 (MMP-7), and cluster of differentiation 24 (CD24).

19. The method of claim 1, wherein the functionally differentiated intestinal cells express mRNA for human lysozyme (LYZ).

20. The method of claim 1, wherein the functionally differentiated intestinal cells express mRNA for a human alpha defensin (DEFA).

21. The method of claim 20, wherein the mRNA for human alpha defensin is mRNA for human alpha defensin 5 (DEFA5) or mRNA for human alpha defensin 6 (DEFA6).

22-26. (canceled)

27. A method of treating graft-versus-host disease, inflammatory bowel disease, Crohn's disease, necrotizing enterocolitis, or intestinal inflammation in an individual in need thereof, the method comprising administering an effective amount of an inhibitor of exportin 1 (XPO1) to intestinal cells of the individual.

28-36. (canceled)
